(19) United States
(12) Patent Application Publication
(10) Pub. No.: US 2024/0058453 A1
(43) Pub. Date: Feb. 22, 2024

(54) Click chemistry hydrogel with minimal swelling as a dural sealant and local delivery vehicle for recombinant proteins and other bioactive agents

(71) Applicants: Virginia Commonwealth University, Richmond, VA (US); Johns Hopkins University, Baltimore, MD (US)

(72) Inventors: Barbara D. Boyan, Henrico, VA (US); Zvi Schwartz, Henrico, VA (US); David J. Cohen, Richmond, VA (US); Jingyao Deng, Richmond, VA (US); David Scott Wilson, Baltimore, MD (US)

(21) Appl. No.: 18/364,891

(22) Filed: Aug. 3, 2023

Related U.S. Application Data
(60) Provisional application No. 63/394,657, filed on Aug. 3, 2022.

(51) Int. Cl. A61K 47/32; A61K 9/06; A61K 38/17; A61K 38/14; A61K 31/7036; A61K 31/05; A61K 47/22; A61K 47/10

(52) U.S. Cl. CPC A61K 47/32; A61K 9/06; A61K 38/1774; A61K 38/14; A61K 31/7036; A61K 31/05; A61K 47/22; A61K 47/10

(57) ABSTRACT

Provided herein is a copper-free, non-toxic click hydrogel that exhibits minimal swelling. The hydrogel may be used as a sealant and/or as a delivery vehicle for local administration of substances of interest, such as therapeutic agents.

### CROSS REFERENCE TO RELATED APPLICATIONS

This application claims the benefit of U.S. Provisional Applications No. 63/394,657, filed Aug. 3, 2022.

### STATEMENT REGARDING FEDERALLY SPONSORED RESEARCH OR DEVELOPMENT

This invention was made with government support under grant number P50 FD004193 awarded by the Food and Drug Administration (FDA) and grant number R01 AR072500 awarded by the National Institutes of Health (NIH). The government has certain rights in the invention.

### BACKGROUND OF THE INVENTION

### Field of the Invention

The invention relates to improved hydrogels for the local delivery of substances of interest. In particular, the invention provides copper-free, non-toxic click hydrogels that exhibit minimal swelling and which may be used for sealing openings such in the dura of mammals (e.g., humans) or for sustained, local in vivo administration of substances of interest, such as therapeutic agents, in or on a selected site of a mammal (e.g., human).

### State of Technology

Hydrogels are formed from precursors that react in situ to produce networks with high water content, imitating the mechanical and chemical properties of surrounding tissues. By altering the quantities and chemical properties of the soluble precursors, it is possible to regulate the mesh size, degradation durations, mechanical properties, and release rates of therapeutic drugs.

Rapidly polymerizing click hydrogels are a method for delivering drugs and biologics to a biological system, as described in issued U.S. Pat. Nos. 10,039,831 and 11,253,597, the complete contents of each of which are hereby incorporated by reference in entirety. However, known hydrogels suffer from problems of a lack of availability of the components and excessive swelling after administration.

It would be of benefit to have available rapidly polymerizing hydrogels and methods of their use that do not suffer from these limitations.

### SUMMARY OF THE INVENTION

The present disclosure provides a non-toxic, physiologically compatible, rapidly gelling hydrogel for which all components are readily commercially available, and which advantageously exhibits minimal swelling compared to prior art gels, a major advantage clinically. The hydrogel also exhibits clinically suitable properties with respect to gelation time, temperature, and degradation. The new formulation had been tested in multiple applications, for example: for delivery of bone inducing agents to stimulate cranial bone formation; for hydrogel delivery of a nerve derived factor, semaphorin 3A (sema3A), to enhance osteogenesis adjacent to a titanium implant in femoral bone in a diabetic model; to enhance osteogenesis adjacent to a titanium implant in femoral bone in a paralysis model; to deliver anti-microbial agents following surgery or trauma, including vancomycin, tobramycin, cefazolin and others; and to deliver anti-inflammatory agents such as resveratrol and specific small RNA, to reduce fibrogenesis during healing; among others.

Accordingly, various embodiments of the present invention are directed to compositions and uses of polymer hydrogels and the delivery of therapeutic agents in vivo. More particularly, various embodiments of the present disclosure are directed to a polymer hydrogel, which is generally formed from a polyacrylate backbone and an alkyne crosslinking member, methods of using the polymer hydrogel, methods of preparing the polymer hydrogel, and kits for making the polymer hydrogel.

Other features and advantages of the present invention will be set forth in the description of invention that follows, and in part will be apparent from the description or may be learned by practice of the invention. The invention will be realized and attained by the compositions and methods particularly pointed out in the written description and claims herein.

In one aspect of the invention, a hole or tear in the dura of a mammal (e.g., the spine or cranial dura of a human) can be sealed by application of a hydrogel having the general formula

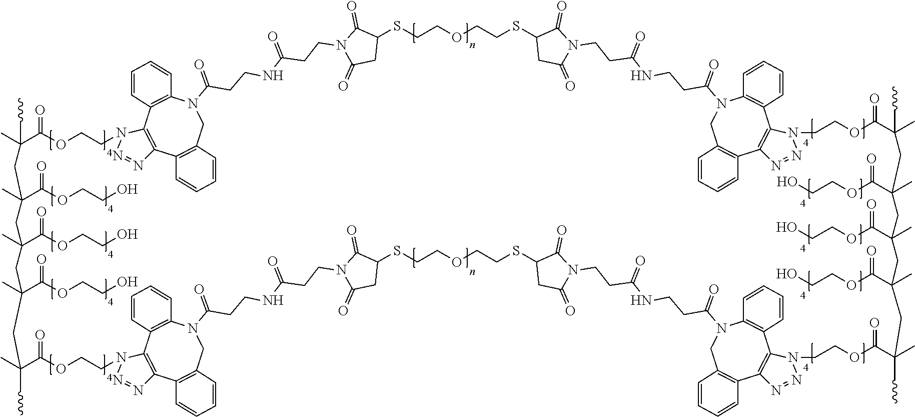

wherein n ranges from 10 to 100 and a wavy line represents a continuation of the polymer backbone. The hydrogel, or similar hydrogels, is preferably formed in place at a selected site by application of an aqueous formulation which includes
   linkers having the formula

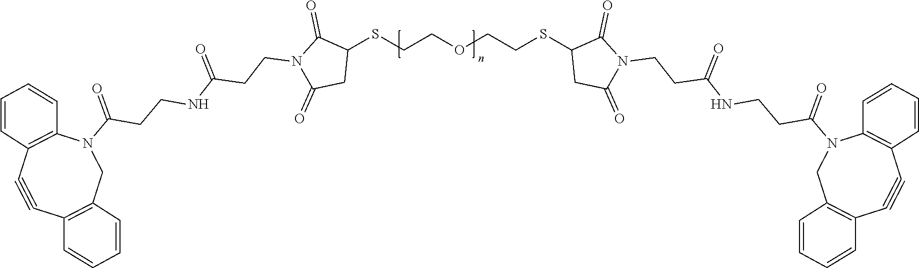

wherein n ranges from 10 to 100, inclusive; and
   water-soluble azide functionalized acylate polymers have the general formula

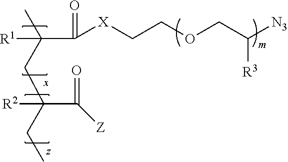

where
R¹ and R² are each independently hydrogen or a C₁ to C6 straight chain, branched chain or cyclic hydrocarbon;
R³ is hydrogen or methyl;
X is —O— or NR⁵— where R⁵ is hydrogen or C₁ to C6 straight chain, branched chain or cyclic hydrocarbon hydrocarbon;
Z is —OR⁶ or NR⁵R⁶ where
   R⁵ is hydrogen or C₁ to C6 straight chain, branched chain or cyclic hydrocarbon, and
   R⁶ is hydrogen, a C₁ to C6 straight chain, branched chain or cyclic hydrocarbon, or a polyethylene glycol chain of two to ten ethylene glycol units;
m is an integer greater than or equal to 1 (e.g. 1-100);
x is an integer greater than or equal to 1 (e.g., 1-100); and
z is zero or an integer greater than or equal to 1 (e.g., 1-100).

The hydrogel and/or the aqueous formulation may be used to treat other selected locations in or on the body of a mammal (e.g., human) by providing the aqueous formulation to the selected site of the mammal. Provisioning of the aqueous formulation may be achieved by injection in, at or on the selected site. In addition, the hydrogel or similar hydrogels may also be used to deliver one or more therapeutic agents at the selected site by incorporating into the aqueous formulation at least one therapeutic agent such as a protein, a nucleic acid, an antibiotic, a polyphenol, a vitamin or a mineral. For example, the therapeutic agent may be one or more of semaphorin 3A, vancomycin, tobramycin, and resveratrol. The selected location may, for example, be a bone fracture in a subject that has diabetes and/or osteoporosis, or a bone fracture in a limb of a subject that is immobile or paralyzed, or a bone implant site, as well as in a joint such as the shoulder, knee, or ankle. The selected location may also include soft tissues, such as tendons and ligaments or non-musculoskeletal tissues, including skin. Recent data show that the formulation can be pre-gelled outside the body and used as a drug delivery device upon implantation. In vitro assays of bacteria killing showed comparable activity by tobramycin in a conventional filter disc to that of tobramycin released from pre-gelled hydrogel disks.

### DESCRIPTION OF THE DRAWINGS

The patent or application file contains at least one drawing executed in color. Copies of this patent or patent application publication with color drawings will be provided by the Office upon request and payment of the necessary fee.

FIGS. 1A and B. Representative images of the ¹H NMR spectra of the individual components of the click hydrogel, the DBCO-functionalized PEG crosslinker (PEG-DBCO) in CDCl₃ (i) and the azide-functionalized RAFT-copolymer (PEG-N3) in D₂O (j).

FIG. 2. Schematic drawing showing polymerization reaction of click hydrogels. The terminal azide on the PEG-N3 reacts with the tri-cyclic alkyne resulting in the polymerized hydrogel (bottom).

FIG. 3A-D. Difference in swelling behavior of the present rapidly-polymerizing click hydrogel compared with the DuraSeal® Dural Sealant System in physiological buffer at 37° C. (A) Swelling response of click hydrogels and the DuraSeal® Dual Sealant System when incubated at 37° C. in PBS over 48 h, defined by the percent increase in horizontal surface area over time. The data are means±SEM via two-way analysis of variance for multiple comparisons with Bonferroni correction *P<0.05 each time point vs. T0, #Duraseal vs. Click Hydrogel at each time point (B) Representative pictures from (A) are shown. Scale bar 5 mm. Vertical swelling capacity of click hydrogels, defined by (C) the percent increase in mass of a hydrogel swelling beyond the constraints of a container and (D) the measured distance from the T0 mark that a hydrogel swells within a container after exposure to PBS at 37° C. for 48 h. Data shown are the means±SEM of three (n=3) independent samples *P<0.05.

FIG. 4A-C. Characterization of hydrogel.

(A): The release of rhBMP2 from the hydrogel was assessed over 10 days. Hydrogels were incubated in 100 uL of media and aliquots were taken at each time point (n=6). (B): Conditioned media experiment to confirm biological activity of rhBMP2 following release. Quantified DNA and osteoblast differentiation markers (n=6). Results of protein ELISAs are normalized to DNA content. (C): Coculture experiment to show rhBMP2 was active immediately following release of hydrogel. Hydrogels were suspended above cell monolayer via well insert. Quantified DNA and osteoblast differentiation markers (n=6) Groups not sharing a letter are statistically significant (p≤0.05).

FIG. 5A-C. Hydrogel Safety Testing. Results of MTT assays to test cytotoxicity of the hydrogel. Media was untreated, or incubated on a polyethylene disc, latex disc, or the hydrogel and then used to treat cells. MTT absorbance assay was used to quantify cell death (n=6). Cell lines examined include A) MC3T3 mouse osteoblasts, B) MRCS human fibroblasts, C) MG63 human osteoblasts.

FIG. 6. Hydrogel Safety Testing. Histology sections of major organs show that hydrogel particles do not impact organ function. Sections taken at 10×, inset at 63×. Groups not sharing a letter are statistically significant (p≤0.05)

FIG. 7A-C. μCT. (A): Representative scans from μCT of experiment groups. Coronal (top), Transaxial (middle) and Sagittal (bottom) planes of each specimen (n=6). (B): 3-dimensional reconstructions of mouse skulls representing each group (Bruker CTVox). (C) Histomorophometrics of μCT from in vivo study showing bone growth. Graphs show bone growth inside the margins of the defect, outside the defect and total bone formation. Groups not sharing a letter are statistically significant (p≤0.05).

FIGS. 8A and B. Histology. (A): Representative histology sections taken from the middle of the defect based on the sagittal plane. Sections imaged at 10×. (B): Quantification of histomorphometrics performed on the sections. Defect closure represents length from new bone growth to the edge of the defect. Total bone growth is a measurement of the area of new bone growth shown in the histology section. Groups not sharing a letter are statistically significant (p≤0.05)

FIG. 9A-J. Physicochemical and rheological properties of rapidly-polymerizing click hydrogels. (A) Squeeze pull-away measurements to assess tackiness of hydrogel samples after 5 min of gelation. The absolute force for tack, time to achieve 90% of force reduction for failure, and area under the curve (N-sec) is indicated to define adhesive and cohesive properties of samples. (B) Frequency sweep performances of the click hydrogel with a fixed strain of 0.5% to determine elastic modulus G′, viscous modulus G″, and phase angle at different frequencies (0.05-20 Hz). (C) Frequency sweep performances indicating log values for complex modulus G* and complex viscosity η* at different angular frequencies. (D) The ratio of elastic modulus G′ to viscous modulus G″ obtained from frequency sweep measurements as a measure of elasticity. (E) Emission intensity values over 5 minutes of the viscosity-sensitive probe, CCVJ, as a measure of gelation. Total fluorescence intensity is measured by the number of pixels in an image taken by fluorescent microscopy at 497 nm above the set threshold value (>1000 A.U.). (F) Swelling response of click hydrogels when incubated at 37° C. in PBS over 48 h, defined by the swelling ratio, qt, or the increase in horizontal surface area over time. (G, H) Vertical swelling capacity of click hydrogels, defined by the percent increase in mass of a hydrogel swelling beyond the constraints of a container, and the measured distance from the T0 mark that a hydrogel swells within a container after exposure to PBS at 37° C. for 48 h. (I, J) Representative images of the 1H NMR spectra of the individual components of the click hydrogel: the DBCO-functionalized PEG crosslinker (PEG-DBCO) in CDCl3, and the azide-functionalized RAFT-copolymer (PEG-N3) in D20. Data shown are the means±SEM of three (n=3) independent samples *P<0.05. For the swelling analyses, groups with * are statistically different at an α=0.05 by one-way ANOVA with Bonferroni post-correction compared to T0. Aside from T0, no significant differences in swelling were detected when comparing individual time points.

FIG. 10A-H. ZDSD rats spontaneously developed diabetic bone phenotype. 15-week-old male Zucker Diabetic Sprague Dawley rats and age-matched Sprague Dawley rats (normal) were put on a high-fat diet until 70% of ZDSD rats turned diabetic. They were then switched to a regular diet. Both groups of rats were aged for 21 days after ZDSD rats turned diabetic and then were assigned to indicated groups: normal GEL, normal 3A+GEL, normal i3A+GEL, diabetic GEL, diabetic 3A+GEL, and diabetic i3A+GEL. Blood glucose levels were measured at indicated time points in normal GEL, normal 3A+GEL, normal i3A+GEL, diabetic GEL, diabetic 3A+GEL, and diabetic i3A+GEL, shown in (A). Body weights were measured on implantation surgery day (B) and animal harvest day (C). The metaphysis of distal femurs (D) was analyzed by 3D microCT reconstructions. Trabecular bone formation was evaluated by Bone Volume/Total Volume (E), Total Porosity (F), Trabecular Thickness (G), and Trabecular Number (H). Data shown are the means for each group ±standard error of n=9 for normal GEL and normal 3A+GEL; n=10 for normal i3A+GEL; n=7 for diabetic GEL and diabetic i3A+GEL; and n=5 for diabetic 3A+GEL for body weights and blood glucose level figures. For trabecular bone phenotype characterization, there were n=8 legs per group. Groups with # are statistically different at an α=0.05 by Student's t-test with Bonferroni post-correction compared to normal groups. Groups with * are statistically different at an α=0.05 by one-way ANOVA with Bonferroni post-correction compared to the corresponding GEL group (B, and C). Groups with @ are significantly different from the normal GEL group at an α=0.05 by unpaired t-test (E-H).

FIG. 11A-K. Sema3A released from hydrogel maintained its bioactivity. Bio-dot assay was conducted over 5 days to determine the release kinetics of sema3A protein from the click hydrogel (A). MG63 cells were cultured on a 24 well plate and treated with vehicle or conditioned media collected from sema3A released from the hydrogel on day 1. The effect of released sema3A on MG63 cell osteoblast differentiation compared to vehicles was assessed by the DNA content (B), production of osteocalcin (C), bone morphogenetic protein 2 (D), osteoprotegerin (E), and osteopontin (F). The comparison to empty hydrogel was assessed by the DNA content (G), osteocalcin production (H), bone morphogenetic protein 2 production (I), osteoprotegerin (J), and osteopontin (K). Data are from a representative experiment and shown as means±standard error of n=6 for each group. Groups with “@” were significantly different from vehicle or empty GEL group using unpaired t-test at an α=0.05. Experiments were performed twice to validate the results.

FIG. 12A-N. Sema3A mitigated the osteopenic bone phenotype in T2DM. 15-week-old male Zucker Diabetic Sprague Dawley rats and age-matched Sprague Dawley rats (normal) were put on a high-fat diet until 70% of ZDSD rats turned diabetic and switched to a regular diet. Both rat groups were aged 21 days after ZDSD rats turned diabetic and then assigned to indicated groups: normal GEL, normal 3A+GEL, normal i3A+GEL, diabetic GEL, diabetic 3A+GEL, and diabetic i3A+GEL. After 28 days of osseointegration, femurs were harvested for microCT scanning. The metaphysis of distal femurs was analyzed by microCT reconstruction (A-F). The trabecular bone phenotype was quantified as Bone Volume/Total Volume (G), Total Porosity (H), Trabecular Thickness (I), and Trabecular Number (J). The cortical bone phenotype was quantified as Bone Volume/Total Volume (K), Total Porosity (L), Cortical Thickness (M), and Mean Cross Sectional Bone Perimeter (N). Data shown are the means for each group ±standard error of n=8 legs/group. Groups with # are statistically different at an α=0.05 by Student's t-test with Bonferroni post-correction compared to normal groups. Groups with * are statistically different at an α=0.05 by one-way ANOVA with Bonferroni post-correction than the corresponding GEL groups. Scale bar=1 mm FIG. 13A-M. Sustained release of sema3A increased marrow BIC and BV/TV. Femurs were harvested for microCT analysis (A-F). Tissue colored brown indicates the cortical bone, and purple tissue indicates the trabecular bone. Total Bone to Implant Contact (G) was analyzed further in the subregions: Marrow Bone to Implant Contact (H) and Cortical Bone to Implant Contact (I). Total Bone Volume/Total Volume (J) were analyzed further in the subregions: Trabecular Bone Volume/Total Volume in the bone marrow (K) and Cortical Bone Volume/Total Volume (L). Qualitative Score on Adjacent Bone (M) was evaluated by qualitative observation of adjacent bone around the implant. Data shown are the means for each group ±standard error of n=8 legs/group. Groups with # are statistically different at an α=0.05 by Student's t-test with Bonferroni post-correction compared to normal groups. Groups with * are statistically different at an α=0.05 by one-way ANOVA with Bonferroni post-correction than the corresponding GEL group. Scale bar=1 mm

FIG. 14A-K. Sema3A increased total BIC regardless of delivery methods. Isolated femurs were fixed in 10% formalin and then embedded in methyl methacrylate. One ground section was taken from each specimen through the center of each implant in a plane longitudinal to the implant and parallel to the long axis of the bone shaft. All sections were stained with Stevenel's Blue/van Gieson stain and cover-slipped (A-F). Osteoid was stained purple and connective tissue was stained blue. Total Bone to Implant Contact (G) was analyzed further into two subregions: Marrow Bone to Implant Contact (H) and Cortical Bone to Implant Contact (I). In addition, Cortical Thickness (J), and Bone Volume/Total Volume (K) were quantified. Data shown are the means for each group ±standard error of n=6 for normal GEL; n=8 for normal 3A+GEL and normal i3A+GEL; and n=6 for diabetic GEL, diabetic 3A+GEL, and diabetic i3A+GEL. Groups with # are statistically different at an α=0.05 by Student's t-test with Bonferroni post-correction compared to normal groups. Groups with * are statistically different at an α=0.05 by one-way ANOVA with Bonferroni post-correction than the corresponding GEL group. Scare bar=500 μm.

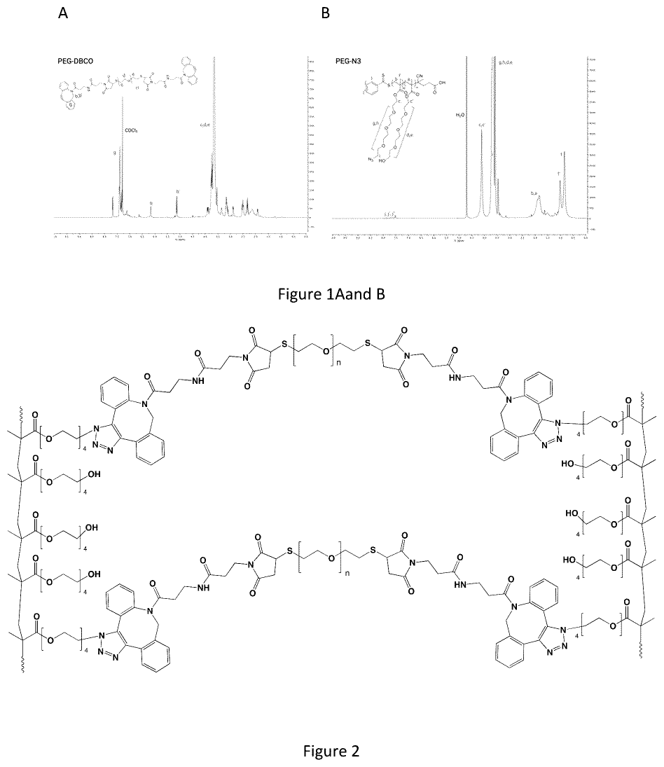

FIG. 15A-D. Sema3A enhanced the bone mechanical properties in T2DM. Maximum Torque (A, C) and torsional stiffness (B, D) were calculated from bilinear curve shown in Supplemental FIG. 1 to evaluate mechanical properties of integrated bone tissues. Data shown are the means for each group ±standard error of n=6 for normal GEL; n=8 for normal 3A+GEL and normal i3A+GEL; and n=4 for diabetic GEL, diabetic 3A+GEL, and diabetic i3A+GEL. Groups with # are statistically different at an α=0.05 by Student's t-test with Bonferroni post-correction compared to normal groups. Groups with @ are significantly different from the normal GEL group at an α=0.05 by unpaired t-test (A, B). Groups with # are statistically different at an α=0.05 by Student's t-test with Bonferroni post-correction compared to normal groups. Groups with * are statistically different at an α=0.05 by one-way ANOVA with Bonferroni post-correction than the corresponding GEL group (C, D).

FIG. 16A-H. T2DM cells do not produce more sema3A on SLA surfaces. Primary osteoblasts were isolated from rat frontal and parietal bones and cultured separately on either TCPS or SLA in DMEM full media. Production of sema3A by osteoblasts from Sprague Dawley rats (A) or diabetic rats (B) was graphed. The effect of exogenous sema3A on rat osteoblast differentiation was assessed as a function of DNA content (C). Production of osteocalcin (D), bone morphogenetic protein 2 (E), osteoprotegerin (F), osteopontin (G), and vascular endothelial growth factor 165 (H) was measured by ELISA of the conditioned media. Data are from a representative experiment and shown as means for each group ±standard error of n=6 individual cultures from pooled rat calvarial osteoblasts. Group with @ are significantly different from the normal GEL group at an α=0.05 by unpaired t-test (A, B). Groups not sharing letters are statistically significant at α=0.05 by one-way ANOVA with Bonferroni post-correction (C—H). Experiments were performed twice to validate the results.

FIGS. 17A and B. Schematic of the experimental procedures. (A) Sprague Dawley rats were divided into 4 groups: BTX and BTX+Sema3A groups underwent botox injection on day 1 and day 25 to ensure the botox effect for the entire study. Veh+Sema3A and BTX+Sema3A groups had sema3A injections on day 21 and day 28. Veh group and BTX group were injected with sterile saline as vehicle controls. Rats were harvested on day 38. (B) Sprague Dawley rats were divided into 6 groups. On day 1, groups 3-6 received 8 units of botulinum toxin type A (BTX) with 2 units to the paraspinal muscles, upper and lower quadriceps, hamstring, and calf. On day 21, groups 1, 3, and 4 received PT implants (smooth), and 2, 5, and 6 received SLAnano implants screwed into the right distal femurs. Groups 4 and 6 were treated with recombinant sema3A delivered via hydrogel in the drilled bone marrow cavity before implant insertion and above the implants after implant insertion. On day 28, botox groups received a second injection of BTX. On day 49, all rats were sacrificed, and femurs were harvested for microCT scanning and removal torque mechanical testing.

FIG. 18A-P. Effect of sema3A on trabecular and cortical bone formation at the distal end of femurs. After 38 days, femurs were isolated and the metaphysis of distal femurs (A) was analyzed with 3D microCT reconstructions: vehicle left femur (B), vehicle right femur (F), veh+Sema3A left femur (C), veh+Sema3A right femur (G), BTX left femur (D), BTX right femur (H), BTX+Sema3A left femur (e), and BTX+Sema3A right femur (i). Trabecular bone volume/total volume (j), total porosity (K), trabecular thickness (1), and trabecular number (M) were quantified from the microCT reconstructions. Cortical bone volume/total volume (N), total porosity (0), and cortical thickness (P) were quantified from the microCT reconstructions. Data shown are the means of treatment/contralateral leg for each group; standard error of n=5 for the vehicle (veh) group, n=8 for the veh+Sema3A group, n=8 for the BTX group, and n=9 for the BTX+Sema3A group. Comparisons between treatment legs and contralateral legs identified with an asterisk, *, are statistically different at α=0.05 by the Wilcoxon matched-paired signed rank test. One-way ANOVA was used for comparisons among multiple groups, and Tukey's post hoc test was used after ANOVA. #p<0.05 vs. veh group, $ p<0.05 vs. veh+Sema3A group. Scale bar=1 mm.

FIG. 19A-O. Effect of sema3A on cortical bone formation at the sema3A injected sites. After 38 days, femurs were isolated, and the sema3A injected sites of distal femurs (A) were analyzed with 3D microCT reconstructions: vehicle left femur (B), vehicle right femur (F), veh+Sema3A left femur (c), veh+Sema3A right femur (G), BTX left femur (D), BTX right femur (H), BTX+Sema3A left femur (E), and BTX+Sema3A right femur (I). Cortical bone volume/total volume (J), total porosity (K), and cortical thickness (1) at the sema3A injection sites were quantified from the microCT reconstructions. Cortical bone volume/total volume (M), total porosity (N), and cortical thickness (0) at the mid-diaphysis were quantified from the microCT reconstructions. Data shown are the means of treatment/contralateral leg for each group standard error of n=5 for the veh group, n=8 for the veh+Sema3A group, n=8 for the BTX group, and n=9 for BTX+Sema3A group. Comparisons between treatment legs and contralateral legs identified with an asterisk, *, are statistically different at α=0.05 by the Wilcoxon matched-paired signed rank test. One-way ANOVA was used for comparisons among multiple groups and Tukey's post hoc test was used after ANOVA. #p<0.05 vs. veh group, $ p<0.05 vs. veh+Sema3A group. Scale bar=1 mm.

FIG. 20A-L. The evaluation of osseointegration by microCT. Male, 12-week-old Sprague Dawley rats were divided into 6 groups: control+PT implants control+SLAnano implants, BTX+PT implants, BTX+PT+Sema3A, BTX+SLAnano implants, BTX+SLAnano+Sema3A groups. Twenty-one days after botox injection, PT or SLAnano were inserted into the distal end of the right femurs. Sema3A was delivered by hydrogel into the bone marrow space before implant insertions and above implants after insertions. Femurs were harvested after 28 days of osseointegration and prepared for microCT scanning. The representative images from microCT were shown in (A) control+SLAnano, (B) BTX+SLAnano, (C) BTX+SLAnano+Sema3A, and the total bone to implant contact (D), bone to implant contact in bone marrow space (E), and cortical bone to implant contact (F) were quantified from 3D microCT images. The osseointegration of PT implants was also evaluated, and the representative images were shown as (G) control+PT, (H) BTX+PT, and (I) BTX+PT+Sema3A. The total bone to implant contact (J), bone implant contact in the marrow space (K), and cortical bone to implant contact (L) were quantified. Data shown are the means for each group +/− standard error of n=8 for control+PT, and control+SLA, n=7 for BTX+SLAnano, and n=8 for BTX+SLAnano+Sema3A, BTX+PT, BTX+PT+Sema3A group. Groups not sharing a letter were significantly different (A vs. B vs. C) by one-way ANOVA and Tukey's post hoc test (α=0.05). Groups that share a letter (A vs. A or AB vs B for example) are not significantly different. Scale bar=1 mm.

FIG. 21A-G. The effect of sema3A on the mechanical properties of rat femurs was assessed by 3-point bending tests, and the mechanical properties of bone around the implants were assessed by removal torque test. Fresh femurs from the first animal study after microCT scanning were prepared for 3-point bending tests, and fresh femurs from the second animal study after microCT scanning was prepared for removal torque mechanical study. Load vs. displacement graph or load vs. radian graph was generated for each femur and fit to a bilinear model (A) to calculate max load (B), stiffness (C), and toughness (D) from a 3-point bending test, and max load (D), torsional stiffness (F), and yield point (G) were calculated from removal torque mechanical test. Data within each group not sharing a letter are significantly different (A vs. B vs. C vs. D for example) at an α=0.05 by one-way ANOVA with Tukey hoc-post correction for multiple group comparison for graphs. Groups that share a letter (A vs. A or AB vs. B for example) are not significantly different (B-D). Two-way ANOVA was used to compare groups in graphs (E-G).

FIG. 22A-D. Difference in swelling behavior of a rapidly-polymerizing click hydrogel compared with the DuraSeal® Dural Sealant System in physiological buffer at 37° C. (A) Swelling response of click hydrogels and the DuraSeal® Dual Sealant System when incubated at 37° C. in PBS over 48 h, defined by the percent increase in horizontal surface area over time. The data are means±SEM via two-way analysis of variance for multiple comparisons with Bonferroni correction *P<0.05 each time point vs. T0, #Duraseal vs. Click Hydrogel at each time point (B) Representative pictures from (A) are shown. Scale bar 5 mm. Vertical swelling capacity of click hydrogels, defined by (C) the percent increase in mass of a hydrogel swelling beyond the constraints of a container and (D) the measured distance from the T0 mark that a hydrogel swells within a container after exposure to PBS at 37° C. for 48 h. Data shown are the means±SEM of three (n=3) independent samples *P<0.05.

FIG. 23. Sealing of dura; volume remaining after 5 minutes.

### DETAILED DESCRIPTION

Provided herein is a copper-free rapidly polymerizing click hydrogel for use as a delivery vehicle for substances of interest. The click hydrogels are physiologically compatible, non-toxic and, advantageously, swelling of the hydrogel in an aqueous environment is minimal. In some aspects, an exemplary polymer hydrogel of the disclosure comprises at least two parts: a water-soluble polyacrylate backbone and a water-soluble alkyne crosslinking agent.

The adaptability of the PEG backbone enables hydrogel applications such as controlled (sustained, long term, etc.) drug release. Further, by adjusting the crosslinker concentration and ratio of various amine functional groups, the mechanical properties of the hydrogel are fine-tuned, including degradation rate, and release kinetics. Significantly, in addition to these benefits, the minimal swelling advantageously enables its usage in tight areas during, for example, spine and cranial surgery, providing a safe approach with a low chance of neurological damage which might otherwise result from gel swelling and undue pressure.

The following definitions are used throughout this disclosure.

### Definitions

As used herein, dura matter refers to the tough outermost membrane enveloping the brain and spinal cord.

As used herein, semaphorin-3A refers to a protein that in humans is encoded by the SEMA3A gene. The SEMA3A gene is a member of the semaphorin family and encodes a protein with an Ig-like C2-type (immunoglobulin-like) domain, a PSI domain and a Sema domain. This secreted semaphorin-3A protein can function as either a chemorepulsive agent, inhibiting axonal outgrowth, or as a chemoattractive agent, stimulating the growth of apical dendrites. In both cases, the protein is vital for normal neuronal pattern development.

As used herein with respect to biology and pharmacology, a small molecule or micromolecule is a low molecular weight (≤about 1000 daltons, e.g., less than about 750 or 500 kDa) organic compound that regulates and/or has an impact on a biological process, Small molecules have a size on the order of 1 nm.

“Precursor solution” refers to an aqueous solution comprising i) water-soluble polymers (typically acrylate polymers) or ii) water-soluble crosslinking molecules (typically alkyne crosslinking molecules), as described herein. The molecules in a polymer precursor solution have not yet reacted to form a gel but will do so upon being combined with a crosslinker precursor solution. In other aspects, a “precursor solution” may refer to a solution comprising both polymers and crosslinkers which exists after rapidly combining the two just before injection, and before gelling.

“Polyphenol” refers to a compound containing more than one phenolic hydroxyl group Polyphenols are a category of compounds naturally found in plant foods, such as fruits, vegetables, herbs, spices, tea, dark chocolate, and wine. Polyphenols are classified on the basis of the number of phenol rings that they contain and of the structural elements that bind these rings to one another. They are broadly divided into four classes: phenolic acids, flavonoids, stilbenes and lignans. Polyphenols are secondary metabolites of plants and are generally involved in defense against ultraviolet radiation or aggression by pathogens. Many polyphenols have antioxidant activity.

The number average molecular weight (Mn) measuring system requires counting the total number of molecules (repeating units) in a unit mass of polymer irrespective of their shape or size so that all molecules in the polymer are treated equally. This is required in cases where certain properties are dependent only upon the number of molecules or repeating units and not upon their weight or sizes, e.g., colligative properties such as boiling point elevation, freezing point depression, vapor pressure depression, and osmotic pressure changes. The number average molecular weight Mn is calculated as

Mn=ΣiNiMiΣiNi

where Mi is the molecular weight of a chain, Ni is the number of chains of that molecular weight, and i is the number of polymer molecules.

“Azide” refers to a linear, polyatomic anion with the formula N-3 and structure —N=N+═N—. It is the conjugate base of hydrazoic acid HN₃.

### Hydrogel Composition

In some aspects, an exemplary polymer hydrogel of the disclosure comprises/is comprised of or formed from at least two parts: a water-soluble polyacrylate backbone and a water-soluble crosslinking agent. In some aspects, the water-soluble polyacrylate backbone is as shown in generic Formula I:

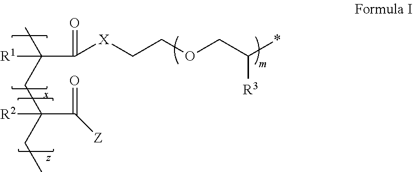

where
R¹ and R² are each independently hydrogen or a C₁ to C6 straight chain, branched chain or cyclic hydrocarbon;
R³ is hydrogen or methyl;
X is —O— or NR⁵— where R⁵ is hydrogen or a C₁ to C6 straight chain, branched chain or cyclic hydrocarbon;
Z is —OR6 or NR⁵R⁶ where
   R⁵ is hydrogen or C₁ to C6 straight chain, branched chain or cyclic hydrocarbon and
   R⁶ is hydrogen; a C₁ to C6 straight chain, branched chain or cyclic hydrocarbon; or a polyethylene glycol chain of two to ten ethylene glycol units;
m is an integer greater than or equal to 1;
x is an integer greater than or equal to 1;
z is zero or an integer greater than or equal to 1; and
* represents a functional group capable of forming a covalent bond with a crosslinking group as described herein. In some aspects, the functional group is azide (an azido group).

In some aspects, when z is greater than zero, the ratio of x to z is between about 5:1 to about 2:1.

In further aspects, m is from 1 to 100 or about 1 to about 10, inclusive, such as about 1, 2, 3, 4, 5, 6, 7, 8, 9 or 10; x is from about 1-50, inclusive, such as about 1, 2, 3, 4, 5, 6, 7, 8, 9, 10, 11, 12, 13, 145, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50; and z is from about 1-50, inclusive, such as about 1, 2, 3, 4, 5, 6, 7, 8, 9, 10, 11, 12, 13, 145, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50.

In some aspects, the water-soluble polyacrylate backbone is a polyacrylate azide of Formula II

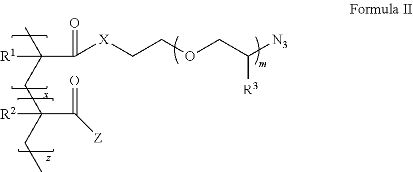

where
R¹ and R² are each independently hydrogen or a C₁ to C6 straight chain, branched chain or cyclic hydrocarbon;
R³ is hydrogen or methyl;
X is —O— or NR⁵— where R⁵ is hydrogen or C₁ to C6 straight chain, branched chain or cyclic hydrocarbon hydrocarbon;
Z is —OR⁶ or NR⁵R⁶ where
   R⁵ is hydrogen or C₁ to C6 straight chain, branched chain or cyclic hydrocarbon and
   R⁶ is hydrogen; a C₁ to C6 straight chain, branched chain or cyclic hydrocarbon; or a polyethylene glycol chain of two to ten ethylene glycol units;

m is an integer greater than or equal to 1;

x is an integer greater than or equal to 1; and

z is zero or an integer greater than or equal to 1.

In some aspects, when z is greater than zero, the ratio of x to z is between about 5:1 to about 2:1.

In further aspects, m ranges from 1 to 100 or is from about 1 to about 10, inclusive, such as about 1, 2, 3, 4, 5, 6, 7, 8, 9 or 10; x is from about 1-50, inclusive, such as about 1, 2, 3, 4, 5, 6, 7, 8, 9, 10, 11, 12, 13, 145, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50; and z is from about 1-50, inclusive, such as about 1, 2, 3, 4, 5, 6, 7, 8, 9, 10, 11, 12, 13, 145, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50.

In some aspects, the polyacrylate backbone is PEG-N3 shown below, where x and z are as described for Formula II and the wavy lines represent a continuation of the backbone chain. A derivatized version is shown in FIG. 1B.

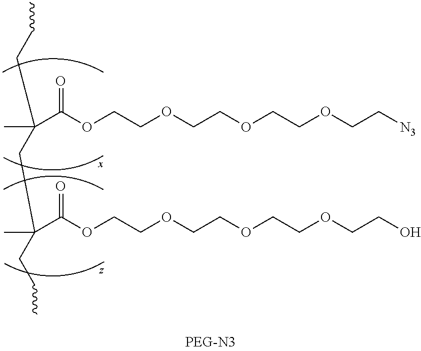

In some aspects, the water-soluble cross-linking agent has general Formula III

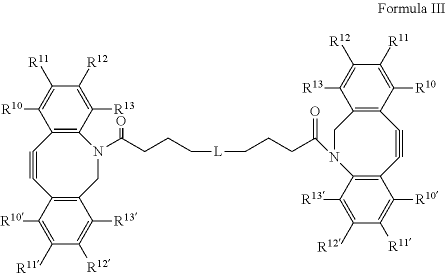

where R¹⁰, R¹¹, R¹², R¹³, R¹⁰′, R¹¹′, R¹²′, and R¹³′ are independently hydrogen, C1 to C6 alkyl, C1 to C6 alkoxy, nitro, fluoro, chloro, or bromo;
and where L comprises the group

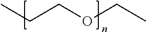

wherein n=10-100, such as 15-95, 20-90, 25-85, 30-80, 35-75, 40-70, 45-65 or 50-60, including all whole integers in between these ranges, i.e., “10-100” represents 10, 11, 12, 13, 14, 15 . . . 95, 96, 97, 98, 99 100. In some aspects, n is about 10, 15, 20, 25, 30, 35, 40, 45, 50, 55, 60, 65, 70, 75, 80, 85, 90, 95 or 100. In some aspect, n ranges from about 65-85.

In some aspects, the crosslinking agent is as shown below

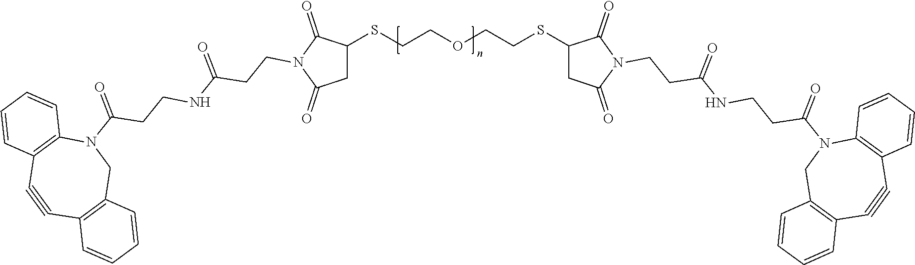

where n=10-100 as described above.

In some aspects, the hydrogel is or is similar to

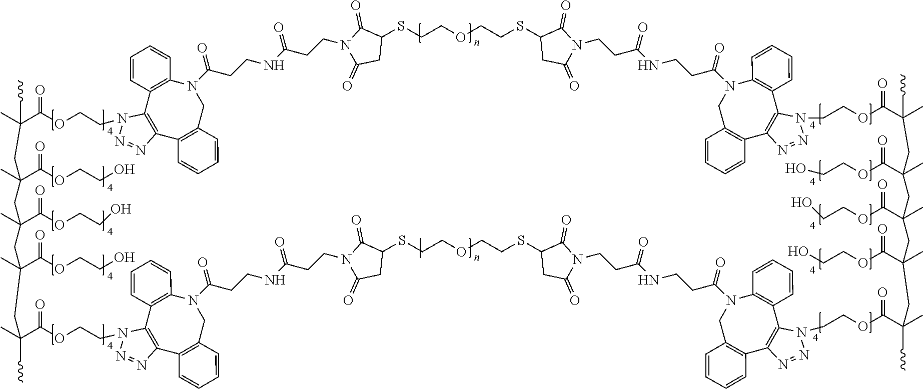

where n=10-100 as described above and each wavy line represents a continuation of the polymer backbone chain within the hydrogel water insoluble three-dimensional network. As can be seen, in this aspect, the azide functional group of a polyacrylate backbone has reacted with a triple bond of the cross-linking agent, converting it to a double bond and forming a triazole ring.

The term “hydrocarbon” as used herein is any branched or unbranched covalently connected series of carbon and heteroatoms, which can be substituted or unsubstituted. The hydrocarbon can be fully saturated or unsaturated, and cyclic or acyclic. Categories of hydrocarbons include alkyls, alkenyls, alkynyls, aryls, and so forth.

A “C₁ to C6 straight chain or branched chain hydrocarbon” as used herein is a branched or unbranched saturated hydrocarbon group of 1 to 6 carbon atoms, such as methyl, ethyl, n-propyl, isopropyl, n-butyl, isobutyl, s-butyl, t-butyl, n-pentyl, isopentyl, s-pentyl, neopentyl, hexyl, and so forth. The alkyl group can also be substituted or unsubstituted. The alkyl group can be substituted with one or more groups including, but not limited to, substituted or unsubstituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein. A “lower alkyl” group is an alkyl group containing from one to six carbon atoms.

The term “cyclic hydrocarbon” (cycloalkyl) as used herein refers to a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. If the cyclic hydrocarbon is substituted, the term “heterocycloalkyl” applies and refers to a cycloalkyl group as defined above and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, substituted or unsubstituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “alkoxy” as used herein is an alkyl or cycloalkyl group bonded through a saturated carbon-oxygen single bond. “Alkoxy” also includes polymers of alkoxy groups as just described; that is, an alkoxy can be a polyether.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, substituted or unsubstituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein. “Cycloalkynyl” includes a cycloalkyl having at least one carbon-carbon triple bond within the ring.

The term “aryl” as used herein is a group that contains any carbon-based aromatic group including, but not limited to, benzene, naphthalene, phenyl, biphenyl, phenoxybenzene, and the like. The term “aryl” also includes “heteroaryl,” which is defined as a group that contains an aromatic group that has at least one heteroatom incorporated within the ring of the aromatic group. Examples of heteroatoms include, but are not limited to, nitrogen, oxygen, sulfur, and phosphorus. The aryl group can be substituted or unsubstituted.

The terms “amine” or “amino” as used herein are moieties having a fully saturated nitrogen with three substituents that are independently, hydrogen or substituted or unsubstituted alkyl, cycloalkyl, alkenyl, cycloalkenyl, alkynyl, cycloalkynyl, aryl, or heteroaryl group described above. The term “carboxylic acid” as used herein is represented by the formula —C(O)OH. The term “halide” as used herein refers to the halogens fluorine, chlorine, bromine, and iodine. The term “hydroxyl” as used herein is represented by the formula —OH. The term “azide” as used herein is represented by the formula —N3. The term “nitro” as used herein is represented by the formula —NO₂. The term “nitrile” as used herein is represented by the formula —CN.

The term “ester” as used herein is represented by the formula —OC(O)— can be a substituted or unsubstituted alkyl, cycloalkyl, alkenyl, cycloalkenyl, alkynyl, cycloalkynyl, aryl, or heteroaryl group described above. The term “amide” as used herein is represented by the formula —N—C(O)—, where the N is fully saturated. The term “carbonate” is represented by the formula —OC(O)O—, the term “carbamate” is represented by the formula —OC(O)N—, and the term “urea” is represented by the formula —NC(O)N—. Species that are alternately substituted at the —O— with an —S— will have the prefix “thio-” as recognized by those of skill in the art.

The term “ether” as used herein is represented by the structural moiety —C—O—C— where each C is independently a carbon of a hydrocarbon, such as a substituted or unsubstituted alkyl, cycloalkyl, alkenyl, cycloalkenyl, alkynyl, cycloalkynyl, aryl, or heteroaryl group as previously described.

The term “polyether” as used herein is a series of repeating ether units that are either the same or different from one another, and having a repeating unit that is an integer of from 1 to 500.

The term “polyglycol” as used herein indicates a category of polyether compounds and includes a repeating chain of substituted or unsubstituted polyethylene glycol units, including polyethylene glycol (PEG) (also called polyethylene oxide or PEO), polypropylene glycol (PPG) (also called polypropylene oxide or PPO) and other substituted polyethylene glycol. The term “glycol” as used herein indicates a subunit of the polyglycol, e.g., polyethylene glycol has a glycol subunit of —CH₂—CH₂—O—.

An acrylate polymer (also known as acrylic or polyacrylate) is any of a group of polymers prepared from acrylate monomers. Acrylates (IUPAC: prop-2-enoates) are the salts, esters, and conjugate bases of acrylic acid. The acrylate ion is the anion CH₂═CHCO₂⁻.

The polyacrylate backbone is generally formed by the polymerization of one or more acrylic acid compounds, such as an acrylic acid, an acrylic ester, an acrylic amide, or the like. The acrylic acid compound may be substituted at any position on the alkene bond by one or more hydrocarbons, such as H₂C═CH—C(O)—, RHC═CH—C(O)—, RR′C═CH—C(O)—, RR′C═CR″—C(O)—, RHC═CR′—C(O)—, or H₂C═CR—C(O)—. Any monomer unit containing an acrylate or di-acrylate may be incorporated into the polymer backbone to generate a multifunctional polymer component of the hydrogel. Examples of suitable acrylate polymers and methods of making the same are found in issued U.S. Pat. No. 11,253,597, the complete contents of which are herein incorporated by reference in entirety.

Those of skill in the art will recognize that by altering the quantities and chemical properties of the water-soluble precursors (backbone and crosslinking components), it is possible to regulate the mesh size, degradation durations, mechanical properties, and release rates of therapeutic drugs that are delivered using the hydrogels.

The composition of an exemplary hydrogel after crosslinking is shown in FIG. 2.

### Methods of Making the Hydrogel

Those of skill in the art will recognize that the methods of making the hydrogel vary depending on the composition and reactive groups of the substituents, i.e., the identity of the polyacrylate backbone and the crosslinking moiety. In general, copper-free click-based chemistry is used to chemically bond two aqueous precursor solutions. Notably, the precursors typically undergo in situ chemical crosslinking, creating a rapidly polymerizing hydrogel at the location at which they are combined.

In some aspects, a thiol-Michel addition reaction involving PEG-dithiol (average Mn 1,000; shown below) where n is a variable number of repeat units commensurate with the average Mn:

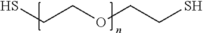

and dibenzocyclooctyne maleimide (DBCO-maleimide) as shown below

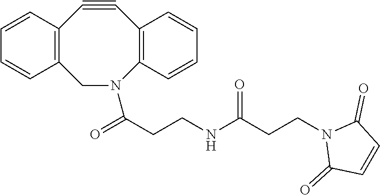 [SMILES: O=C(CCN1C(=O)C=CC1=O)NCCC(=O)N1Cc2ccccc2C#Cc2ccccc21]

are used to create a poly-ethylene glycol (PEG) crosslinker that has been functionalized with dibenzocyclooctyne (DBCO). In some aspects, reversible addition-fragmentation chain transfer (RAFT) polymerization, which allows for the precise control of azide functionality, is used to create PEG-N3 from azide functionalized and non-functionalized PEG methacrylate monomers to produce a water-soluble, non-fouling multivalent azide functionalized acrylate polymer. When an aqueous solution of this crosslinker is combined with an aqueous solution of this azide functionalized acylate polymer, an in situ crosslinked hydrogel forms rapidly.

Generally, the gelling time of the hydrogel ranges from about 15 to about 120 seconds at 37° C. and is typically from about 30 to about 100 seconds, such as about 30, 40, 50, 60, 70, 80, 90, 100, 110, or 120 seconds. In some aspects, the gelling time is 90 seconds or less.

In order to form a hydrogel in situ from two aqueous solutions, it is necessary to mix the two solutions rapidly just before placing the mixture at the desired location, or, alternatively, to place the solutions separately but substantially simultaneously at the desired location, such as on a bone or adjacent to a bone (usually immediately adjacent with little or no intervening tissue), or at the site of a wound. Given the typical volumes of solutions that are used, placement is generally accomplished by injection using a needle.

As discussed herein, in some aspects, the hydrogels disclosed herein are used to deliver at least one substance of interest to a location of interest. The at least one substance of interest is typically, but may not always be, a therapeutic agent. The at least one substance of interest is generally compatible with the hydrogel precursor formulations, e.g., is soluble in or can be dissolved or uniformly suspended in an aqueous solution of a hydrogel precursor.

The substance of interest may be added to (mixed with) one or both of the aqueous precursor polymer and crosslinker solutions before injection, or with a combined precursor solution comprising both polymers and crosslinkers before injection (mixing must be followed by immediate administration to prevent gelling in the delivery device), or the substance of interest may be injected separately but substantially simultaneously at the same location at which the precursor polymer and precursor crosslinker solutions are (separately) injected. For example, all solutions must be injected within less than 90 seconds, such as within about 60 seconds or less, such as about 50, 45, 40, 35, 30, 25, 20, 15, or 10 seconds or less if possible.

### Characteristics of the Hydrogel

The hydrogels disclosed herein advantageously exhibit minimal swelling after gelation (i.e., after initial crosslinking of the acrylate polymers occurs) under physiological conditions and/or in aqueous media. While initial crosslinking occurs within seconds as described above, the hydrogel generally swells (e.g., increases in volume and surface area) for several hours, such as for 2-24 hours, e.g., for about 2, 4, 6, 8, 10, 12, 14, 16, 18, 20, 22 or 24 hours.

The swelling ability of a hydrogel after gelation can be measured by changes in surface area, mass, and volume of the hydrogel between steady-state and upon suspension in a physiological buffer. In particular, for changes in surface area, the ratio of horizontal swelling and the ratio of vertical swelling are each typically calculated.

For the hydrogel formed from the polyacrylate backbone PEG-N3 and the crosslinking agent PEG-DBCO, after submersion in physiological buffer for 24-48 hours, the average horizontal swelling ratio generally ranges from about 1.0 to 8.5, such as about 1.0, 1.5, 2.0, 2.5, 3.0, 3.5, 4.0, 4.5, 5.0, 5.5. 6.0, 6.5, 7.0, 7.5, 8.0 or 8.5. Typically, the average horizontal swelling ratio is less than about 5.0, such as about 4.5, 4.0, 3.5, 3.0, 2.5, or 2.0; and is frequently about 2.0, 2.1, 2.2, 2.3, 2.4, 2.5, 2.6, 2.7, 2.8, 2.9 or 3.0.

For the hydrogel formed from the polyacrylate backbone PEG-N3 and the crosslinking agent PEG-DBCO, after submersion in physiological buffer for 24-48 hours, the ratio of vertical swelling generally ranges from about 5.0 to 25.0, such as about 5.0, 5.5, 6.0, 6.5, 7.0, 7.5, 8.0, 9.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, 20.0, 20.5, 21.0, 21.5, 22.0, 22.5, 23.0. 23.5, 24.0, 24.5 or 25.0.

The increase in mass (after 24-48 hours in physiological buffer) generally ranges from about 15-20%, such as about 15, 16, 17, 18, 19 or 20% (see FIG. 3C).

The increase in volume (after 24-48 hours in physiological buffer) generally ranges from about 25-35%, such as about 25, 26, 27, 28, 29, 30, 31, 32, 33, 34 or 35% (See FIG. 3D).

Overall, the % swelling was about 100% (see FIG. 3A).

The polymer hydrogel disclosed herein generally contains a substantial amount of water after gelling, as is typical of hydrogels. This water content provides the polymer hydrogel with the ability to deliver therapeutic agents to a location while still maintaining its structure and characteristics. In one aspect, the polymer hydrogel has a concentration in water of between about 0.5% and 25%, generally between about 1% and about 20%, or between about 1.5% and about 15%, and or between about 1.5% and about 12.5%, all expressed in w/v of the polymer hydrogel in water. Thus, the amount of water can be, for example, about 0.5, 1.0, 2.0, 3.0, 4.0, 5.0, 6.0, 7.0, 8.0, 9.0, 10.0, 11.0, 12.0, 13.0, 14.0, 15.0, 16.0, 17.0, 18.0, 19.0, 20.0, 21.0, 22.0, 23.0, 24.0 or 25.0 percent, including all decimal fractions in between these valued, to 0.1 decimal places (e.g., 0.5, 0.6, 0.7, 0.8, 0.9. 1.0 . . . 24.5, 2, 24.6, 24.7, 24.8, 24.9 and 25.0 percent)

### Exemplary Methods of Using the Hydrogel

The rapid gelation and polymerization of the polymer hydrogel of the instant invention advantageously permits its use to treat areas of the body in need of immediate, watertight sealing, with or without simultaneous delivery of a therapeutic agent. For example, the hydrogel adsorbs fluids, including blood, making it a useful adjunct for wound sealing and controlling hemorrhaging.

These properties also advantageously permit the in situ delivery of therapeutic agents by the hydrogel. Thus, in some aspects, the hydrogels, when used, comprise at least one (i.e., one or more) therapeutic agents therein. In some aspects, the therapeutic agents include agents which are used for medical and/or cosmetic treatments and/or reconstruction in biological systems.

Exemplary therapeutic agents comprise biologically active agents which include but are not limited to: macromolecules such as proteins; peptides; nucleic acids (examples of which include DNA; RNA such as microRNA (shRNA, siRNA, etc.), exosomes; “small molecule” drugs; active agents that occur naturally or are derived from “natural products” such as polyphenols, an example being resveratrol; cells; minerals such as calcium; and vitamins such as vitamin D.

In an exemplary embodiment, the hydrogel composition contains a protein or nucleic acid. In some aspects, the composition contains a protein. The composition can contain a protein that is between about 1 to 1000 kDa, or about 5 to 500 kDa, or about 10 to 100 kDa, or about 25 to about 50 kDa, or about 15 to about 35 kDa.

In an exemplary aspect, the therapeutic agent(s) is/are provided for use as a liquid solution. The liquid solution comprising at least one therapeutic agent is miscible with a polyacrylate precursor solution and/or a crosslinker precursor solution. comprising both (or a precursor solution which comprises one) of the components that form the polymer hydrogel prior to covalent bonding of the two hydrogel components, or at least prior to complete covalent bonding of the two hydrogel components, i.e., prior to gelling in situ. Thus, as discussed above, the therapeutic agents may be mixed with one or both of the hydrogel components while the components are in liquid form. Alternatively, the therapeutic agent(s) and the hydrogel components are mixed together all at once (substantially simultaneously), for example, just prior to or at the same time the liquid gel components are injected at a site of interest. Thus, administration involves the simultaneous delivery (e.g., injection of) of at least one and possibly two or three (or more) solutions to/at the site of interest. The therapeutic agent(s) is/are thus encapsulated in, absorbed suspended in, dissolved in, intercalated within or even chemically linked (covalently or non-covalently) to the polymer hydrogel, or a combination of these. The therapeutic agent may or may not be chemically bonded to the polymer hydrogel. Accordingly, the therapeutic agent(s) is/are delivered in the polymer hydrogel to a specific location of the body where gelling occurs so that the polymer hydrogel localizes the delivery of the therapeutic agents to that specific location.

The hydrogels function to carry biologically active agents to a selected location and then advantageously release them in situ. Release can be over an extended period of time. Accordingly, the disclosure provides methods of delivering a therapeutically effective dose of one or more (at least one) therapeutic agent (medicament) to a subject in need thereof, at a location within the subject at which the therapeutic agent(s) act to prevent or treat at least one symptom of a disorder or malady and/or to increase the effectiveness of another therapeutic modality. A therapeutic dose is generally any amount of an agent that prevents or alleviates at least one symptom of the disorder or malady. Those of skill in the art will recognize that the exact amount that is delivered depends on several factors, such as the gender, age and overall health of the subject, whether or not other medicaments are being administered, the seriousness of the disorder or malady, etc. In general, the amount of the therapeutic agent that is delivered is in the range of from about 0.001 to about 1000 mg/kg of subject weight, such as about 0.001, 0.01, 0.1, 1.0, 10, 20, 30, 4, 50, 60, 70, 80, 90, 100, 150, 200, 250, 300, 350, 400, 450, 500, 550, 600, 650, 700, 750, 800, 850, 900, 950 or 1000 mg/kg, including all decimal fractions (e.g. 1.0, 1.1, 1.2, 1.3 . . . etc.), integers (e.g. 10, 11, 12, 13, 14, 15 . . . etc.) in the range between each listed value. Those of skill in the art will recognize that while in some cases administration may result in complete prevention or alleviation of at least one symptom of the disorder/malady, much benefit can be derived from only lessening or decreasing at least one symptom. For example, pain and/or swelling may be lessened, the rate of healing may be increased, infection or the risk of infection may be decreased or eliminated, the quality of healing may be improved such as the strength of a bone, the adherence of bone to an implant, the rate of bone deposition at a site of interest, a decrease or prevention of fibrosis, more rapid and/or more complete healing of dural tears and bone fractures, etc.

After setting of the hydrogel, an initial burst release of the therapeutic occurs, usually during the first 24 hours after delivery of the hydrogel. Generally, the initial burst provides a concentrated dose of the agent(s), such as about 25-85 percent of the payload, i.e, about 25, 30, 35, 40, 45, 50, 55, 60, 65, 70, 75, 80 or 85%. Preferably, the initial burst, which generally occurs over 1-2 days, is less than about 75%, or less than 50%. Thereafter, the remaining agent is released at a steady, gradual rate as the hydrogel degrades. Degradation and slow, gradual release generally occurs over a period of, for example, one month (i.e., about 1, 2, 3, or 4 weeks), or about 30 days, such as about 2, 3, 4, 5, 6, 7, 8, 9, 10, 11, 12, 13, 14, 15, 16, 17, 18, 19, 20, 21, 22, 23, 24, 25, 26, 27, 28, 29 or 30 days. In some aspects, slow release occurs over about 3 weeks or over about 2 weeks, or over about 1 week, or over about 10-14 days. The hydrogel thus provides sustained release of the therapeutic agent(s) delivery thereby.

The present hydrogel technology may be used to deliver therapeutics to sites which include but are not limited to areas: just beneath the skin, in and around joints, next to bones and implants, adjacent to scars and scar tissue, adjacent to teeth (e.g., into periodontal pockets or at or near dental implants), at or adjacent to tears or breaks in tissue and bones, at or near any type of wound, at or near tears or holes in the brain and/or spinal dura, or at or near surgical incisions.

The hydrogels disclosed herein are used to deliver therapeutic agents to prevent and/or treat a wide variety of diseases and conditions. Examples include but are not limited to: the prevention of resynostosis following suturectomies for craniosynostosis; the prevention of fibrosis after orthopedic surgery (e.g. by delivering an agent such a resveratrol that inhibits production of TGFβ1 and activation of collagen synthesis by fibroblasts); for the treatment of dural tears, e.g., in the skull due to injury, surgery, birth defects, etc., such as the delivery of morphogenetic protein 2 to enhance closure and healing); to enhance bone regeneration, including adjacent to implants (such as delivering an osteoinductive protein such as the small peptide, semaphorin 3a, especially in the elderly or in diabetic patients in whom bone formation is attenuated); to treat long bone nonunions; to deliver agents to “tight” confined locations or areas in need of a therapeutic agent (such as in or adjacent to the spine, joints, skull, etc.) without causing mechanical injury to the underlying tissues; and for the treatment of wounds, both accidental and surgical (e.g., to prevent fibrosis following orthopaedic surgery).

In certain aspects, since the click hydrogel exhibits substantially less swelling than other currently available hydrogels, the present hydrogels are especially useful to successfully deliver agents to confined locations without causing mechanical injury to the underlying and/or surrounding tissues. An example includes their use as dural sealants.

In some aspects, the hydrogels are used to deliver agents to treat or prevent bone injuries, e.g., to treat a condition in, at, on, within or near a bone. For example, bone fractures are treated. In some aspects, the fractures are treated by the placement of an implant. In other aspects, the fractures are treated by immobilization and/or casting (hard or soft cast). In further aspects, the limb in which the fracture (or fractures) is/are treated is immobile, immobilized, paralyzed (fully or partially), or the patient is otherwise unable to exert pressure on the limb (e.g., cannot walk) so that normal wright bearing movement is prevented or attenuated. In these situations, the hydrogels can advantageously deliver therapeutic agents to the location/area of the break. Thus, the hydrogels deliver agents that: increase (enhance) bone formation and/or growth; inhibit (decrease) bone resorption; decrease bone porosity; increase trabecular thickening; increase the amount of bone attached to an implant; increase osteoblast differentiation; increase parameters such as maximum torque and torsional stiffness; increase marrow BIC and BV/TV; etc. Exemplary agents which facilitate or bring about these effects (e.g., bone healing, repair, and/or regeneration) are known in the art and include but are not limited to: semaphorin 3A, calcium, vitamin D and its metabolites, bisphosphonates, strontium ranelate, teriparatide (a synthetic form of a parathyroid hormone), etc.

Exemplary methods of using the hydrogel include but are not limited to: delivery of an antibiotic (e.g., vancomycin) for applications in spine surgery; delivery of an antibiotic (e.g., tobramycin) for applications in orthopedic and spine surgery; delivery of resveratrol to reduce inflammation following joint surgery (e.g., hip, knee, or finger joint replacement); delivery or anti-inflammatory agents to reduce or prevent fibrosis; delivery of anti-inflammatory agents to reduce or prevent tendon fibrosis; delivery of anti-inflammatory agents to reduce or prevent lung fibrosis; delivery of anti-inflammatory agents and/or analgesics to treat arthritis; delivery of lipid nanoparticles (LNPs) to deliver agents such as small RNA in situ in orthopaedic and neurosurgery applications; delivery of microRNA to promote cartilage defect healing; sustained delivery of therapeutic agents in dural sealants; sustained delivery of analgesics following shoulder surgery; sustained delivery of osteogenic biologics like BMP2 to promote peri-implant bone growth; sustained delivery of antimicrobial compounds following trauma sustained release of osteogenic biologics like BMP following cranial bone surgery; sustained release of osteogenic agents to promote peri-implant bone formation around dental implants; etc.

In some aspects, the hydrogels are used in oral surgery and/or treatment procedures. In this aspect, use of the hydrogel meets clinical needs which include but not limited to:
treatment of periodontal disease (e.g., treatment of periodontal pocket) by delivery of one or more antibacterial agent sand/or delivery of one or more anti-bone resorption agents;
regeneration of bone (e.g., before, during and/or after periodontal periodontics, trauma treatment or surgery, treatment of genetic defects, surgery involving tumors, etc.);
regeneration of tooth material after implant insertion, where the hydrogel delivers agents before, during and/or after the implant is placed.

In this context, types of treatment include but are not limited to:
1. Sustained delivery of antimicrobial compounds to treat periodontal disease.
2. Sustained release of osteogenic biologics that induce bone formation/regeneration, such as a bone morphogenetic protein (BMP).
3. Sustained release of osteogenic biologics that inhibit bone resorption, for example, anti-RANKL agents such as anti-RANKL antibodies (e.g., denosumab, monoclonal antibody JMT103) and/or the natural inhibitor osteoprotegerin (OPG), etc.).
4. Sustained release of osteogenic agents to promote peri-implant bone formation around dental implants.

In some aspects, the hydrogel is utilized to deliver positive or negative effectors, e.g., inhibitors (negative) or activators (positive), of the activity of various RNAs, such as microRNAs. The effects exerted by these agents may be direct or indirect. For example, effectors of various microRNAs, including but not limited to: microRNA 2, miR-21-5p, miR-29a-3p, miR-29b-3p, miR-29c-3p, miR-let-7a, miR-663a, miR-122, miR-133b, miR-149-5p, miR-126, miR-124, miR-132, miR-9, miR-25, miR-27a, miR-30a, miR-9, miR-31, miR-34a, miR-145, miR-146a, miR-146b-5p, miR-148a, miR-155, miR-193b, miR-203, miR-335, miR-497, miR-34c, miR-200c, miR-574-5p, miR-671-5p, miR-181b, etc., are delivered. The effectors include mimics and inhibitors of the miRNAs. An example of an inhibitor of microRNA 2 is resveratrol. The hydrogel containing such agents may be used for the treatment of cancer and metastasis, e.g., with surgical intervention or separately.

In other aspects, effectors, e.g., inhibitors or activators, of the activity of transforming growth factor beta (TGFβ) are delivered using the hydrogel. For example, the treatment of a dysfunction of TGF-β1 signaling (which has been implicated in several human diseases, including cancer, cardiovascular diseases, fibrosis, atherosclerosis and developmental defects) can be prevented or treated by delivering suitable agents with the hydrogel.

Examples of anti-inflammatory agents that are delivered using the hydrogel include but are not limited to: steroids such as cortisone, hydrocortisone and prednisone; and non-steroidal anti-inflammatory drugs (NSAIDs) such as aspirin, ibuprofen, naproxen, diclofenac, celecoxib, etc.

Examples of analgesics that are delivered using the hydrogel include but are not limited to: non-opioid analgesics, opioid analgesics, and compound analgesics that combine the two previous forms. Non-opioid analgesics include but are not limited to acetaminophen and nonsteroidal anti-inflammatory drugs (NSAIDs) such as: ibuprofen, aspirin, naproxen, naproxen sodium, naproxen/esomeprazole, diclofenac, etodolac, indomethacin, nabumetone, oxaprozin. Examples of compound analgesics include but are not limited to: co-codamol, co-codaprin and co-dydramol. Examples of opioid analgesics include but are not limited to:

codeine, fentanyl, hydrocodone, meperidine, methadone, morphine, oxycodone and tramadol.

Accordingly, methods of administering, to a subject in need thereof, precursors of the hydrogels disclosed herein are provided. The methods generally include a step of administering, generally by injection, at least one biocompatible liquid solution comprising the precursors of the hydrogel to a specific site or location in the subject. The precursor solution(s) contain acrylate polymers, which may be azide derivatized acrylate polymers, and alkyne crosslinking agents as described herein. In some aspects, the subject who is treated has suffered a bone fracture. In some aspects, the subject is scheduled to receive, is in the process of rece3iving (i.e., is in surgery for), or has already received an implant as a result of the fracture. In some aspects, the limb where the fracture occurred is immobile or immobilized. The subject may have health issues that predispose him or her to fractures and/or slow or difficult healing of fractures, examples of which include advanced age, diabetes, or osteoporosis.

### Exemplary Kits

Also provided herein are kits for the treatment of an anatomical part of a body. In some aspects, the kits include an aqueous solution of a polyacrylate azide as disclosed herein, an aqueous solution of a crosslinking alkyne as disclosed herein, and, optionally, an aqueous solution of a therapeutic agent. In other aspects, the kits may include dry or concentrated forms of the precursors and buffer solutions suitable for adding to the concentrated solutions to form precursor solutions suitable for administration.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

In the description of the invention herein, it is understood that a word appearing in the singular encompasses its plural counterpart, and a word appearing in the plural encompasses its singular counterpart, unless implicitly or explicitly understood or stated otherwise. Furthermore, it is understood that for any given component or embodiment described herein, any of the possible candidates or alternatives listed for that component may generally be used individually or in combination with one another, unless implicitly or explicitly understood or stated otherwise. Moreover, it is to be appreciated that the figures, as shown herein, are not necessarily drawn to scale, wherein some of the elements may be drawn merely for clarity of the invention. Also, reference numerals may be repeated among the various figures to show corresponding or analogous elements. Additionally, it will be understood that any list of such candidates or alternatives is merely illustrative, not limiting, unless implicitly or explicitly understood or stated otherwise. In addition, unless otherwise indicated, numbers expressing quantities of ingredients, constituents, reaction conditions and so forth used in the specification and claims are to be understood as being modified by the term “about.”

Accordingly, unless indicated to the contrary, the numerical parameters set forth in the specification and attached claims are approximations that may vary depending upon the desired properties sought to be obtained by the subject matter presented herein. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the subject matter presented herein are approximations, the numerical values set forth in the specific examples are reported as precisely as possible. Any numerical values, however, inherently contain certain errors necessarily resulting from the standard deviation found in their respective testing measurements.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

All patents and publications mentioned in the specification are indicative of the level of those skilled in the art to which the invention pertains. All patents and publications are herein incorporated by reference in their entirety to the same extent as if each individual publication was specifically and individually indicated to be incorporated by reference.

### EXAMPLES

### Example 1. Newly Formulated Hydrogels Exhibit Reduced Swelling

Hydrogels are formed from precursors that react in situ to produce networks with high water content, imitating the mechanical and chemical properties of surrounding tissues. By altering the quantities and chemical properties of the soluble precursors, it is possible to regulate the mesh size, degradation durations, mechanical properties, and release rates of therapeutic drugs. In an exemplary aspect, the polymer hydrogel comprises at least two parts, a polyacrylate backbone as shown in FIG. 1A and a crosslinking member shown in FIG. 1B. In some aspects, the polyacrylate backbone and the crosslinking member are connected directly or via an intervening substituent (FIG. 2).

The swelling of the newly formulated click hydrogel and DuraSeal® Dural sealant were compared. The two compositions were incubated at 37° C. in physiological buffer (PBS) for 48 hours. The swelling ability was measured by the change in surface area, mass, and volume of the hydrogel between steady-state and upon suspension in a physiological buffer. The results are presented in FIG. 3A-D.

### Example 2. Rapidly Polymerizing Click Hydrogel Provides for Localized Delivery of rhBMP2 to Promote Bone Formation

### Introduction

The goal of this study was to examine if the novel click hydrogel could serve as an improved alternative to traditional methods of rhBMP2 delivery. We hypothesized that our hydrogel could provide sustained, localized delivery to an injury site better than a collagen scaffold. Our first step was to try and reduce the burst release of rhBMP2 away from the defect, minimizing the chances of the protein stimulating bone formation in soft tissues and outside the target area of healing. The second step was to ensure that the release of the protein was sustained over a longer period of time in the hope that as the hydrogel degraded and released protein, a lower overall dose would be required to stimulate new bone formation, thereby minimizing the risks surrounding high-dose levels of rhBMP2. To this end, we confirmed the release profile and characteristics of rhBMP2 from our novel hydrogel and ensured its non-toxicity, and then used a critical sized calvarial defect model in juvenile mice to assess its potential as a drug delivery vehicle for rhBMP2.

### Materials & Methods

### Hydrogel Material Preparation

To generate our rapidly polymerizing hydrogel, PEG-N3 was synthesized from azide functionalized and non-functionalized PEG methacrylate monomers through reversible addition-fragmentation chain transfer polymerization, resulting in tight control of azide functionality. The generated polymer had a molecular weight of approximately 25 kDa with an average azide functionality per polymer of 13. A difunctionalized PEG-DBCO crosslinker was synthesized by reacting bis-amino-PEG with excess benzyl-2-nitro-carbonate functionalized DBCO. Recombinant bone morphogenetic protein 2 (rhBMP2) is a component of the TGF beta signaling pathway shown to be important in the development of cartilage and bone and induces osteoblast differentiation in a variety of cells.

### In Vitro Hydrogel Characterization

All in vitro experiments were performed under aseptic conditions. Aqueous stock solutions of PEG-DBCO (12.5%; w:v) crosslinker and PEG-N3 (50%; w:v) were prepared by vortexing and sonicating the polymers in PBS at room temperature. Two parts PEG-DBCO and 1 part PEG-N3 were incubated on ice until they were mixed by pipetting. Hydrogels were then placed in the bottom of 200 μL Eppendorf tubes containing 100 μL DMEM full media. At each time point, 10 μL of media was removed and replaced with 10 μL fresh media. Results were quantified using a rh-BMP2 ELISA (RND Systems; DY355) The biological activity of rh-BMP2 delivered from the 12.5% w:v hydrogel was performed by incubating gels containing 10 ng rh-BMP2 at 37° C. for 2 days in DMEM. The conditioned media, 20 ng/mL rhBMP-2 or full media were added to MG63 cells at 80% confluence. After 48 hours, cells were harvested and tested for markers of osteoblastic differentiation.

In order to test release of the protein directly to the cells, tissue culture polystyrene plates with well inserts were used (Thermo Fisher Scientific; 141002). Hydrogels were loaded with 10 ng rh-BMP2 or without protein. Two parts PEG-DBCO and 1 part PEG-N3 were pipetted onto the insert and mixed. MG63 cells were cultured to 80% confluence on TCPS and inserts were placed into each well for 48 hours then harvested. Hydrogel groups and the negative controls were treated with DMEM full media, and the positive control received DMEM full media supplemented with 20 ng/mL rh-BMP2.

### In Vitro Hydrogel Safety Testing

Aqueous stock solutions of PEG-DBCO (12.5%; w:v) and PEG-N3 (50%; w:v) were prepared by vortexing and sonicating the polymers in PBS at room temperature. Two parts PEG-DBCO cross linker and 1 part PEG-N3 were incubated on ice until mixing by pipetting. Media was incubated in Eppendorf tubes containing 4.69 mm diameter latex, high density polyethylene disks or hydrogel for 24 hours. MC3T3, MRC-5, and MG-63 cells were grown to 80% confluence on a 96-well TCPS plate. Cells were treated with conditioned media from the Eppendorf tubes or unconditioned media that had been in an empty Eppendorf tube for 24 hours.

Media were replaced with fresh serum free DMEM. An MTT assay was performed by adding methylthiazolyldiphenyl-tetrazolium bromide (Sigma-Aldrich; M2128) stock solution to the wells. After 4 hours, wells were aspirated and DMSO was added to dissolve the formazan crystals that had been produced. Following a 15-minute incubation, wells were read for their absorbance.

### Intracutaneous Injection of Hydrogel Extract for In Vivo Safety

Extraction fluid was prepared following ASTM Standard F619-03: Standard Practice for Extraction of Medical Plastics. Two parts of PEG-DBCO (12.5%; w:v) click hydrogel crosslinker was combined with 1 part PEG-N3 (50%; w:v) to form a 1.6 g hydrogel. This was combined with 5 mL 0.9% NaCl solution in a sterilized boroscillate glass container and placed in a water bath at 37° C., capable of agitation. The container was observed for signs of mixing and removed from the water bath then shaken vigorously for 30 seconds before decanting the extract liquid into a sterile container.

Male New Zealand White Rabbits were prepared for injection by shaving a large area of the back on both sides of the spinal column providing for a sufficient test area. Loose hair was removed by means of a vacuum and the skin was sterilized with alcohol swabs. Extract liquid was agitated prior to withdrawal of each injection dose. Rabbits were injected intracutaneously with 0.2 mL of hydrogel extract at 5 sites on the same side of the animal. 0.2 mL of 0.9% NaCl blank extract solution was injected at 5 sites on the opposite side of the back. The injection sites were examined at 24, 48 and 72 hours for gross evidence of tissue reaction, such as erythema, edema, or necrosis. Scoring was based on tables from the ASTM Standard F749-13: Standard Practice for Evaluating Material Extracts by Intracutaneous Injection in the Rabbit.

### Guinea Pig Maximization Test

Extraction fluid was prepared in the same way as above, following ASTM Standard F619-03: Standard Practice for Extraction of Medical Plastics. Test samples were prepared for intradermal injection by combining 0.05 mL Freund's complete adjuvant with 0.05 mL 0.9% NaCl, 0.05 mL extract with 0.05 mL 0.9% NaCl and 0.05 mL Freund's complete adjuvant with 0.05 mL extract. Mixtures were homogenized by continuous vortex for 5 minutes. Animals were shaved at the shoulder region exposing a 4×6 cm area and 3 injection sites were chosen at least 1.5 cm apart. One week following injections the area was re-shaven and treated with 10% sodium lauryl sulfate (SLS) in petroleum jelly 24 hours prior to applying test patches. Test sample was mixed with petroleum jelly and applied to a 2×4 cm filter paper until saturated. The filter paper was then placed on the injection site and secured with occlusive surgical tape and an elastic bandage for 48 hours. Two weeks later a 5×5 cm area was shaved on the animals' flanks and filter paper saturated in the test agent was applied to the animal in the same manner for 24 hours. After removing patches test sites were examined at 1 hour, 24 hours, and 28 hours for signs of erythema and edema. Scoring was done in accordance with ASTM Standard F720-81: Standard Practice for Testing Guinea Pigs for Contact Allergens: Guinea Pig Maximization Test and the allergenicity of the hydrogel was determined.

### In Vivo Critical Defect Study

All critical sized calvarial defects were created 45-day old male C57Bl/6J mice. A 1 mm midline incision was made 5 mm posterior to the eyes. Under 28× magnification the interfrontal ridge, posterior frontal suture and bregma were identified before creating the defect. The defect was made using a 4 mm internal diameter trephine drill, 1 mm lateral to the posterior frontal suture, centered between the interfrontal ridge and coronal sutures. The defects were left empty, filled with a collagen sponge containing 1 μg of rhBMP2 in sterile 1×PBS or injected with 16 μL of the 12.5% empty hydrogel or those containing 1 μg of rhBMP2. The ratios and mixing were performed as described above and the polymerization was verified after 20s with a blunt 25G needle. All mice were randomized to both treatment group and post-operative time. Defects were randomized to contain 2 mL of: empty defect, hydrogel only, hydrogel with 1 ug of rhBMP2. One group of mice were euthanized immediately following surgery to serve as a control for defect size. Bone healing in and around the defect was assessed after 28 days using μCT reconstruction and analysis. Histological assessment was performed by hematoxylin and eosin (H&E) staining of decalcified 7 mm axial sections in the middle of the defect and analyzed by light microscopy.

### MicroCT Analysis

Calcified skulls were scanned using μCT to assess bone formation. Specimens were fixed to specimen holders in a Skyscan 1172. Scanning was performed at 57 kV and 87 mA. The rotational step and zoom was set at 0.2 degrees and 20.14 μm voxel size, respectively, and a 1 mm aluminum filter was used to reduce noise, creating a 1024×1024 pixel image matrix. Scans were processed and beam-hardening reduction was performed at 20%, and ring artifact reduction and post alignment were carried out at different steps based on the quality of the scanned image. The histogram setting was set to a constant range of 0 to 0.038834. Scans were reconstructed and analyzed using CTan software by selecting a VOI to create a binary representation of bone versus defect to measure defect area and bone volume.

### Histomorphometric Analysis

After imaging with mCT the samples were thawed and the brain was removed taking care not to damage the defect. The samples were fixed in 10% neutral buffered formalin, changing the solution after 24 h. The skulls were decalcified by immersing samples in Calfor® decalcification solution (formic acid and EDTA) for a period of 72 hours. Complete de-calcification was verified by slicing through the rostrum. Under 4× magnification the center of the defect was visualized and a coronal cut was made through the center of the defect. The samples were dehydrated with ethanol and embedded in paraffin. Sections 7 mm in thickness were made and stained with H&E using standard protocols. The samples were imaged at 10× magnification. The average defect width and area of bone formation in and around the defect were calculated (Fiji; ImageJ).

### Statistical Analysis

All data are represented as the mean±standard error of the mean. The sample size for all in vivo and in vitro experiments was determined by a prospective power analysis based on previously reported data. All cell culture experiments were performed with six independent cultures (n=6) and repeated two times. All in vivo experiments were performed with at least 6 mice per group (n=6). For all in vitro and in vivo experiments, a one-way ANOVA was performed and significance among groups was determined by a multiple comparison test with Tukey adjustments. Statistical significance for all experiments was declared when the p-value was less than 0.05.

The Virginia Commonwealth University Institutional Animal Care and Use Committee approved animal procedures in accordance with the guide for the Care and Use of Laboratory Animals.

### Results

### Hydrogel Release Kinetics

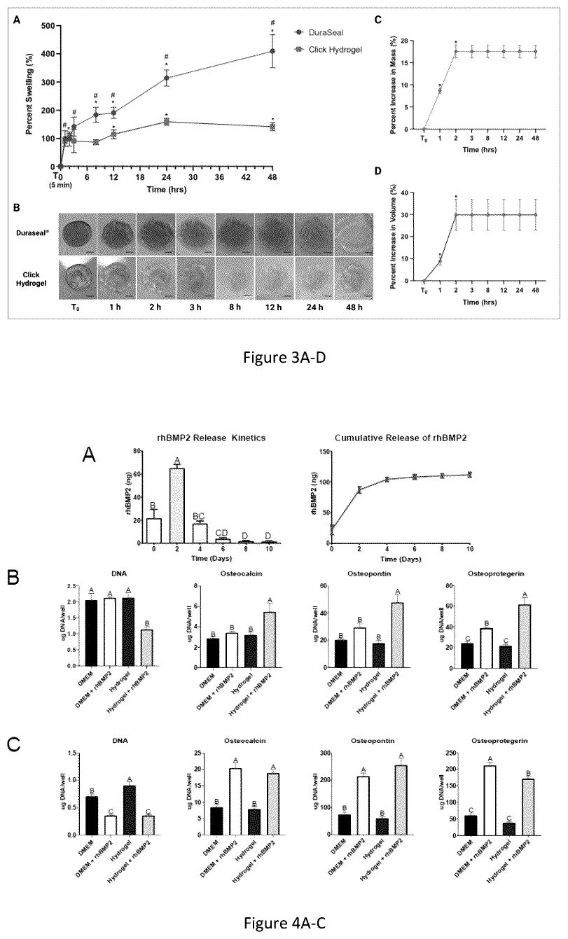

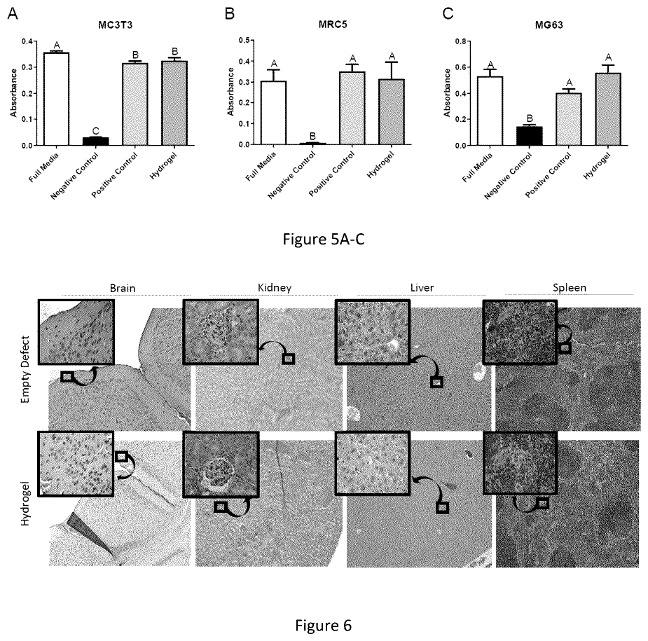

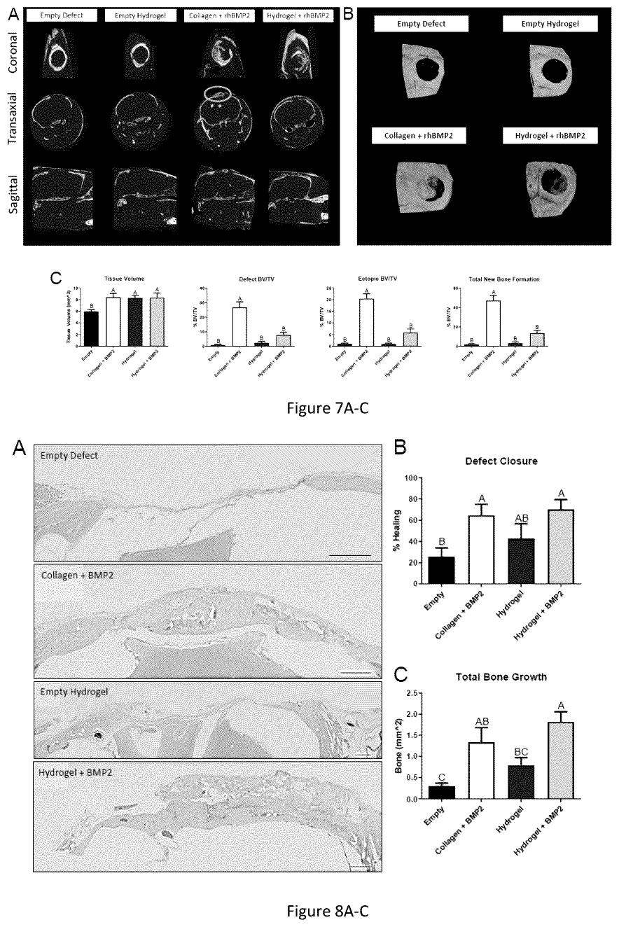

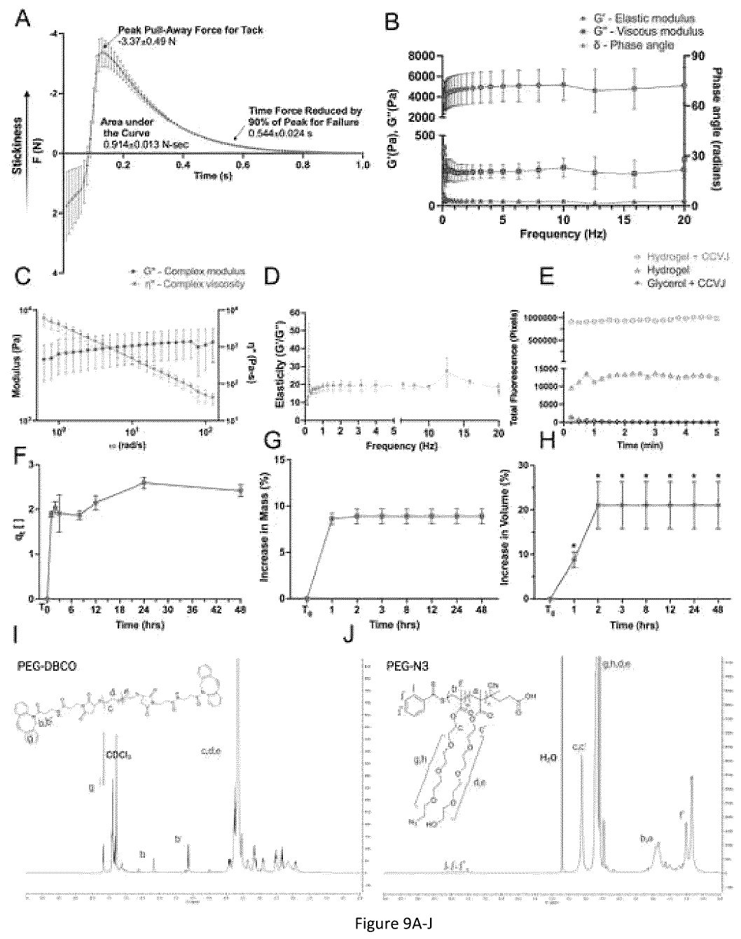

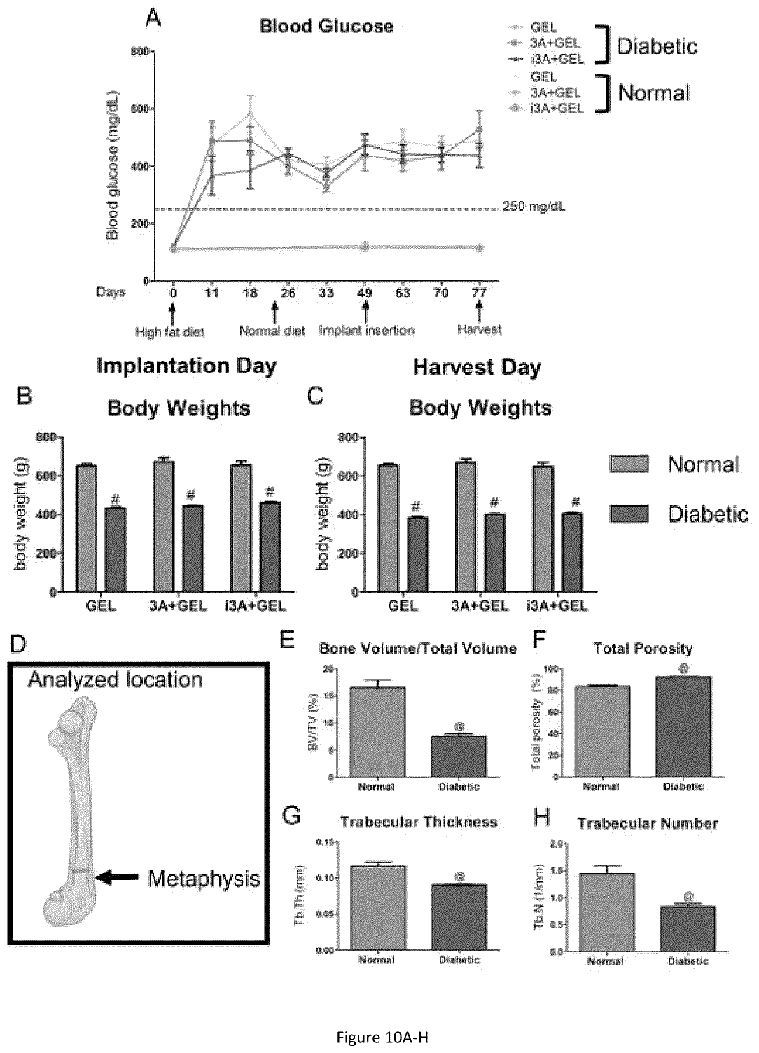

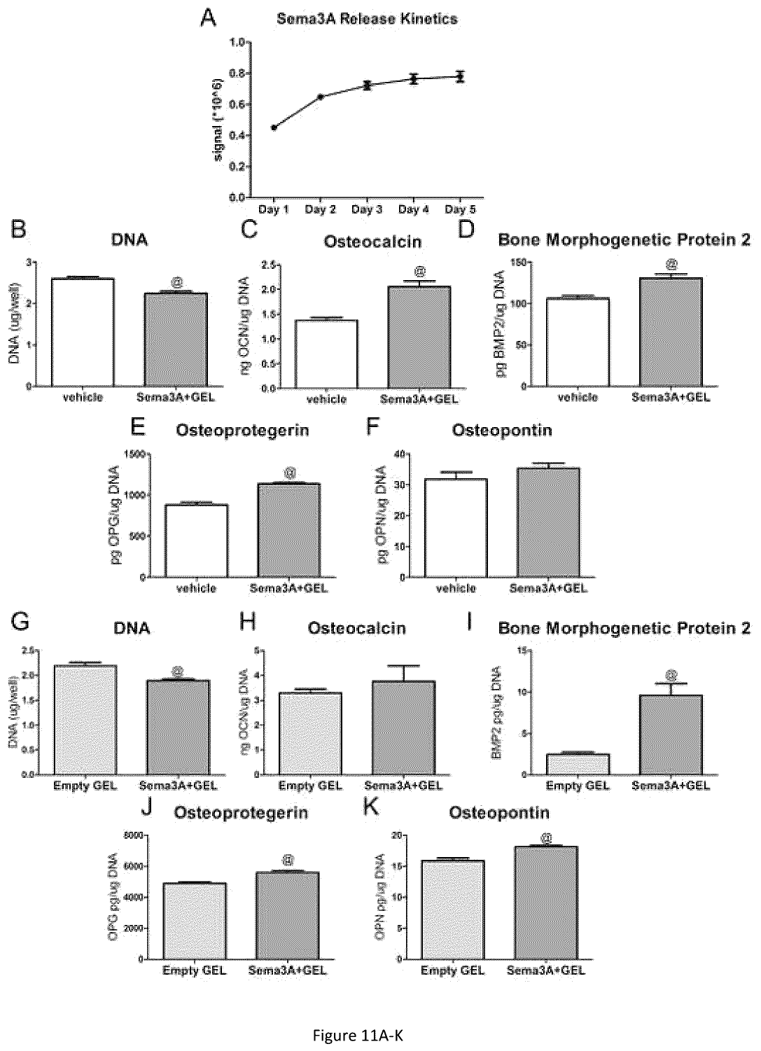

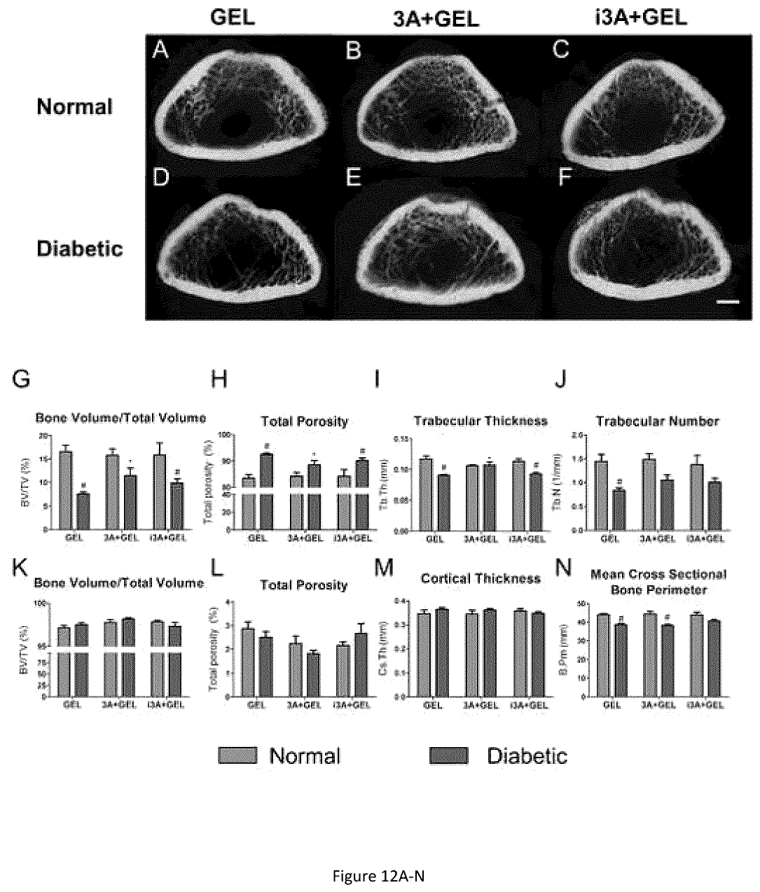

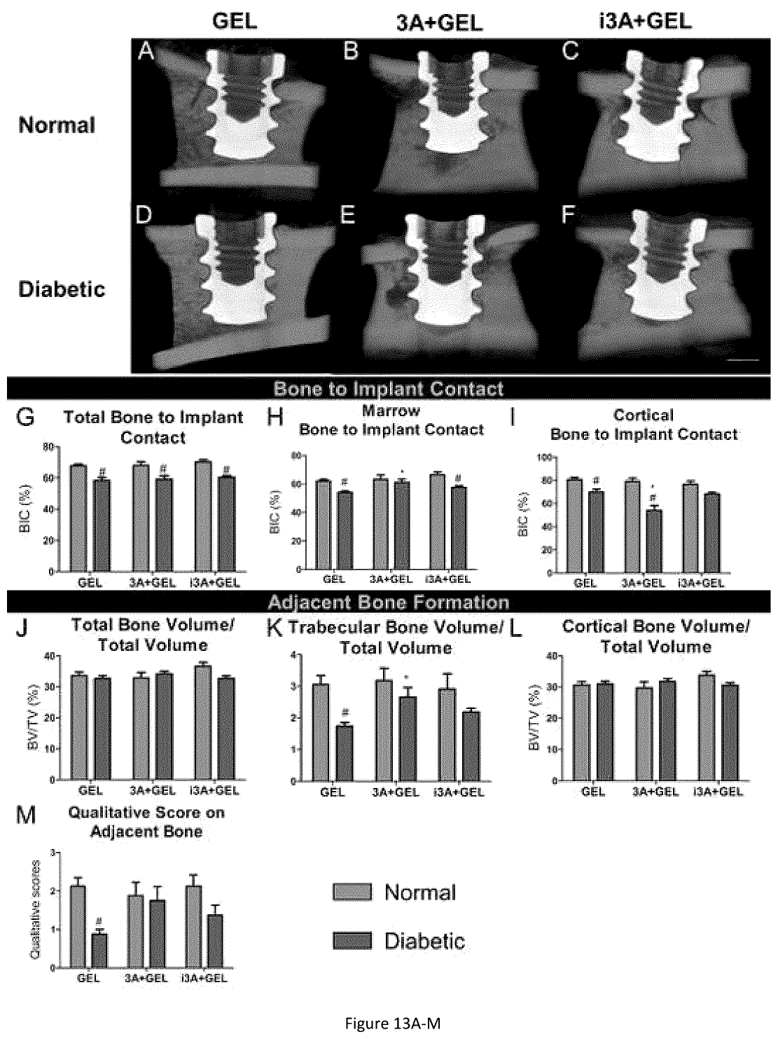

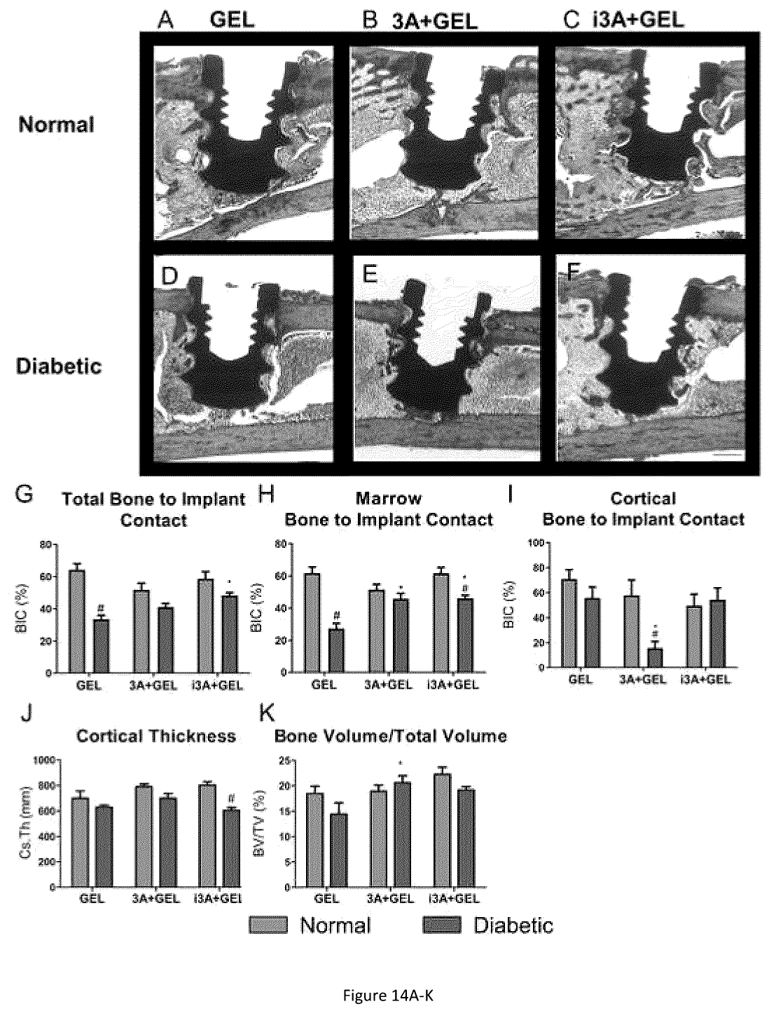

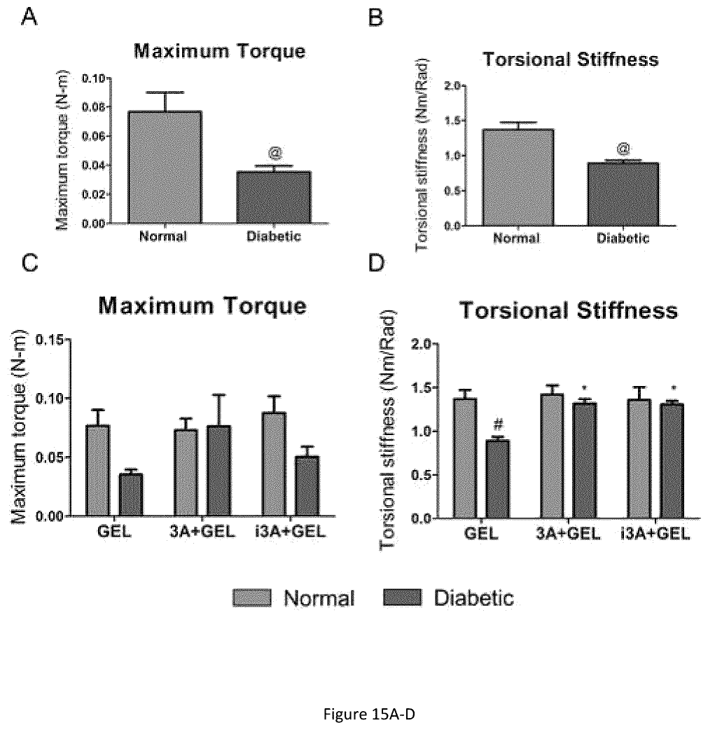

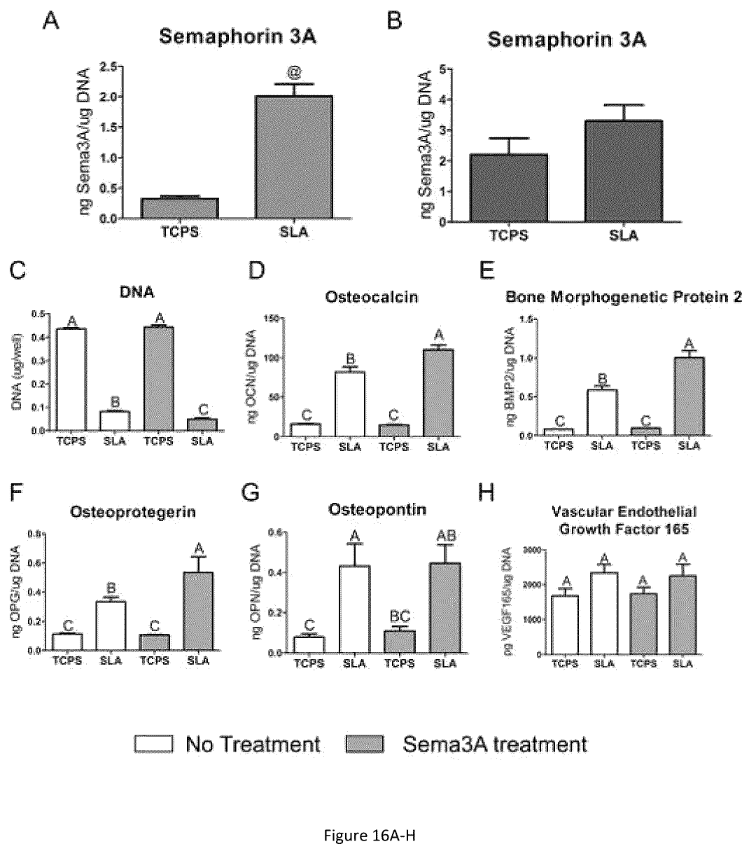

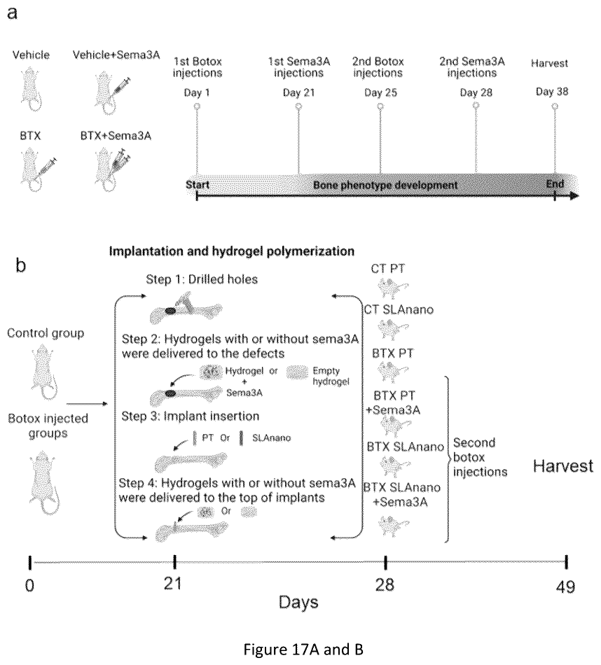

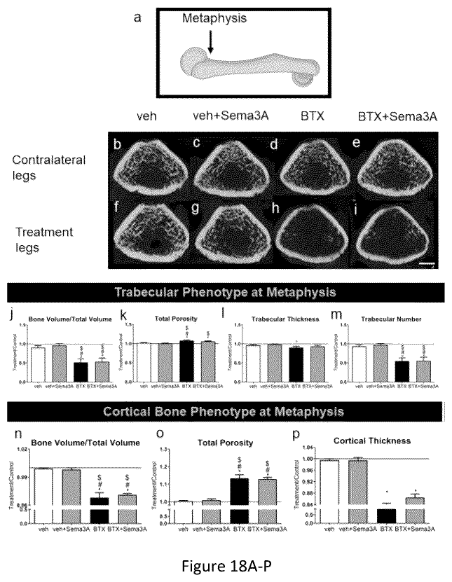

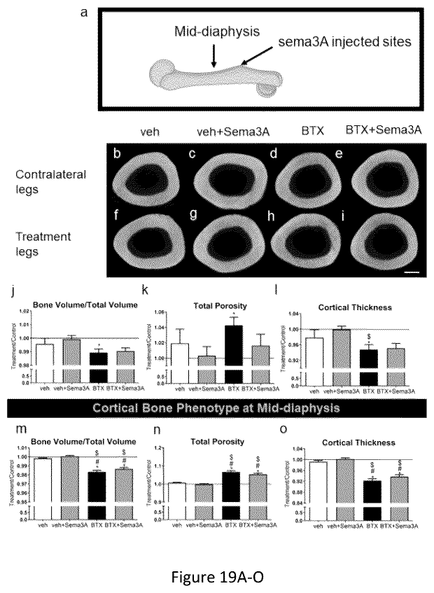

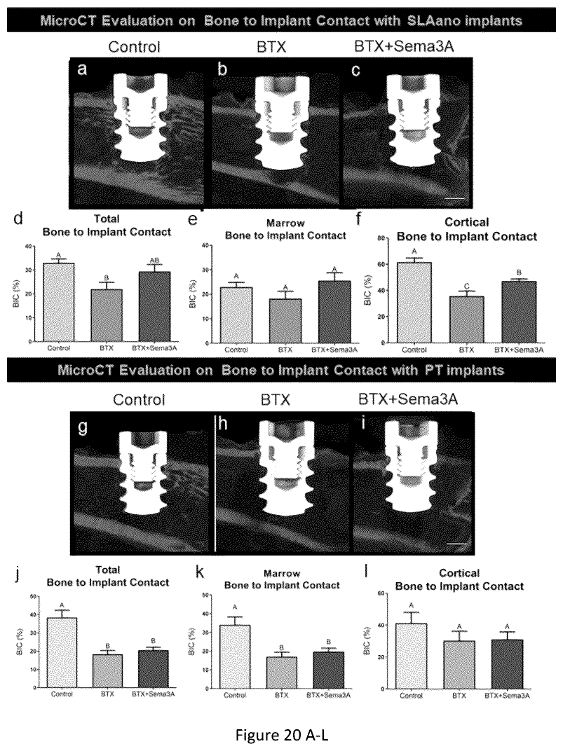

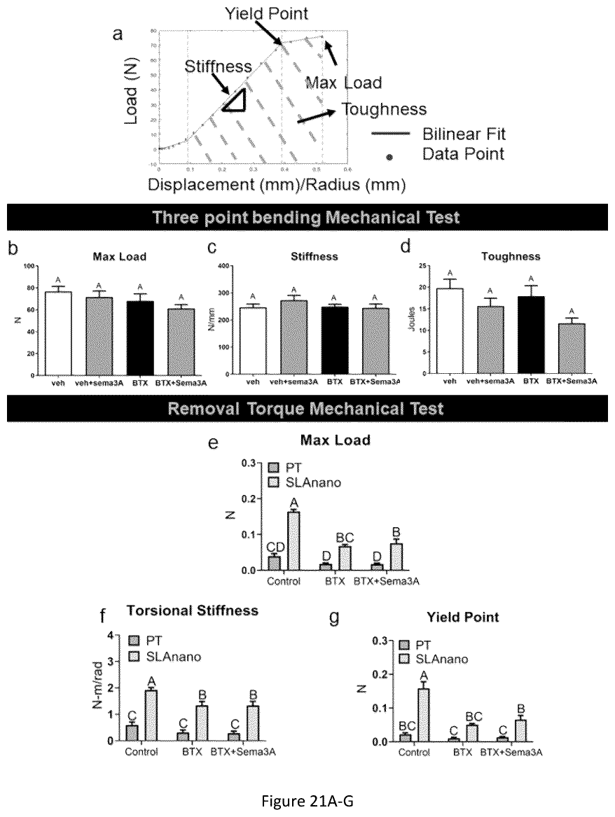

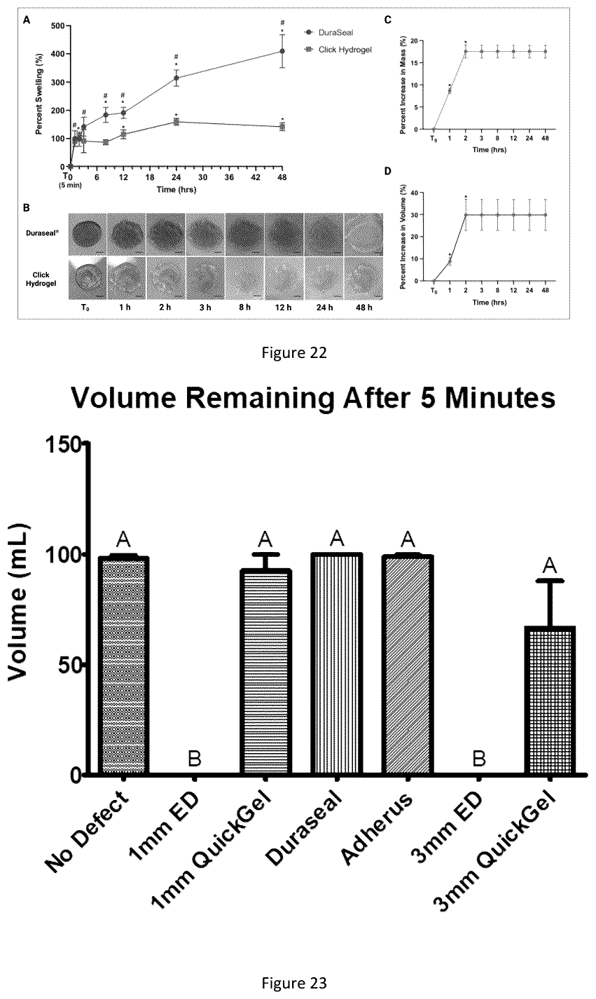

The release of rhBMP2 from the hydrogel showed a consistent sustained release (FIG. 4A). Results were quantified using a Human BMP-2 ELISA (R&D Systems DY 355-05). A 12.5% w:v crosslinker was chosen to form the hydrogel based on previous results that showed a more prolonged and consistent release compared to other ratios. Results show an initial burst release between days 0 and 2 with a large burst occurring on day 2. By the end of day 2, 87% of the rhBMP2 had been released from the hydrogel. The remaining rhBMP2 was then slowly released over the following 8 days, with a sustained gradual decrease through 10 days until no rhBMP2 remained in the hydrogels.

Bioactivity of rhBMP2 Release from Hydrogel

Two experiments were performed to ensure that the biological effect of the rhBMP2 was preserved after being released from the hydrogel. First conditioned media that had been exposed to the hydrogel containing rhBMP2 was used to treat osteoblast progenitor MG63 cells. The cells treated with rhBMP2 released from the hydrogel showed signs of less proliferation, as well as an increase in osteoblastic differentiation markers osteopontin, osteocalcin and osteoprotegerin (FIG. 4B).

A transwell culture system was used to further confirm the bioactivity of the rhBMP2 released from the hydrogel. Hydrogels containing rhBMP2 were placed on a porous membrane suspended above the well plate surface and allowed to release the protein into the media over 48 hours. The resulting effect showed that enough rhBMP2 was released from the hydrogel to match the effect of the positive control that had rhBMP2 supplemented in the media. Both test groups showed a decrease in proliferation and an increase in differentiation as exhibited by heightened amounts of osteopontin, osteocalcin, and osteoprotegerin (FIG. 4C).

Hydrogel in vitro Safety Testing

An MTT assay was used to examine if the hydrogel were cytotoxic to cells and if the lack of proliferation experienced by the MG63 cells were a result of the rhBMP2 or cell death. Three cell lines were tested, all of which showed that the hydrogel was not cytotoxic. In each case the high-density polyethylene disc used as a positive control matched the measured absorbency of the hydrogel group (FIG. 5A-C). This confirmed that the differentiation of the MG63 cells were a result of the biologically active rhBMP2 rather than cell death being falsely viewed as a sign of decreased proliferation.

Hydrogel in vivo Safety Testing

The major organs of mice were excised, sectioned and examined for evidence of gross morphological changes. In particular, histology sections were examined for hydrogel particulate to ensure that as the hydrogel degraded in the body, the matter did not circulate and appear in any of the major organs (FIG. 6).

In the rabbit subcutaneous sensitization test, the injection sites on the rabbits showed very little signs of erythema and edema 24 hours after injection. After 48 and 72 hours, there were no signs of edema or erythema on any of the injection sites. It can be posited that any sign of erythema in the first 24 hours after injection was a result of injury to the superficial capillaries by the needle during injection rather than irritation caused by the hydrogel material. Likewise, the sensitization allergen test performed on guinea pigs showed no signs of response to the hydrogel extract in either vehicle when compared to the Mercaptobenzothiazole control.

In vivo Calvarial Defect Study

Mice were sacrificed 4 weeks after placement of the hydrogel to assess bone formation. The tissue volume shows that the region of interest selected around the defect was consistent across all groups that received a treatment. Analysis was performed using Bruker CTan and bone inside the defect was measured by drawing a region of interest within the margins of the defect. The amount of bone inside the defect was normalized to tissue volume to examine how effectively the defect closed over 4 weeks. The collagen sponge with rhBMP2 showed a significant amount of bone growth, and although it was not significant compared to the control, there is a similar trend for the bone growth in the hydrogel with rhBMP2 group (FIG. 7A-C). The ectopic bone was measured as any bone growth outside the defect not consistent with the regular morphology of the parietal bone. The hydrogel with rhBMP2 showed the presence of some bone growth outside the margins of the defect, but it was not significant compared to the control. Evidence of this can be seen in the cross-sectional views of the defect (FIG. 7A-C). The collagen sponge with rhBMP2 showed a significant amount of bone growth outside the defect. A majority of the animals had a mass of bone protruding outside the defect as shown in the cross section, as denoted by the circle (FIG. 6). The empty hydrogel had very little effect on bone growth compared to the control. None of the animals across test groups exhibited complete closure of the defect. Three-dimensional reconstructions of microCT scans confirm the results of the quantification. There was little to no healing in the empty defect and empty hydrogel groups. The hydrogel with rhBMP2 showed the presence of some bone growth in the defect, while the collagen with rhBMP2 group shows the mass of ectopic bone growth on the left side above the defect (FIG. 7C)

### Histology Section of Calvarial Defect

Histology sections were taken from the middle of the defect based on the sagittal plane. Representative images are shown (FIG. 8A) Evidence of bone growth can be seen in both the hydrogel and collagen with rhBMP2, while the empty hydrogel and empty defect show only thin lines of connective tissue spanning the defect. Histomorphometric analysis was performed using ImageJ software. Defect closure was based on the length measured from the margin of the defect to new bone growth and presented as percent healing. Both the collagen and hydrogel with rhBMP2 were significant to the empty defect (FIG. 8B). Total bone growth was measured as the area of new bone growth shown in the section both inside and outside the margins of the defect. The area of new bone formation for the hydrogel loaded with rhBMP2 was significant compared to the empty hydrogel and empty defect. The collagen sponge with rhBMP2 showed a trend of increased bone formation and was significant compared to the empty defect, but not when compared to the empty hydrogel (FIG. 8C).

### Discussion

Our results demonstrate that the present click hydrogel is a viable method of delivery for rhBMP2 when used as a therapeutic for bone fracture healing. The release profile of our hydrogel showed that although the hydrogel does experience a burst release 48 hours after injection, it continues to release protein over the course of 10 days. This can be considered a similar release profile to the absorbable collagen sponge that shows an initial burst release followed by a gradual release with a retention of less than 5% the initial dose after 2 weeks. Moreover, the present hydrogel demonstrated its ability to carry rhBMP2 and retain its biological activity in vitro. The increased production of osteogenic markers meant that the hydrogel had no effect on the rhBMP2 when it was encapsulated, proving it can be an alternative carrier to the absorbable collagen sponge.

The safety of the hydrogel both in vitro and in vivo was also important. By testing its cytotoxicity on a range of cell lines, we were able to confirm that contact between the hydrogel material and tissue would be safe and not produce inflammation or an apoptotic effect. The hydrogel was tested on two types of pre-osteoblast cell lines, one for humans and one for mice, because the primary site of injection would be in and around bone. Neither of which were harmed by the hydrogel, and as a way of further confirming its innocuous nature, we tested it on a soft tissue MRC-5, human lung tissue cell line, which showed no adverse effects to the hydrogel.

The first step of in vivo testing was to ensure that the hydrogel did not produce a reaction dermally or intracutaneously. This was accomplished by following the ASTM standards for testing sensitization of injected materials. In both the rabbit intracutaneous and guinea pig dermal sensitization tests the hydrogel groups produced very little if any erythema or edema reinforcing the results of the in vitro testing that the hydrogel was non-inflammatory. Another concern was the degradation of the hydrogel and where the material would possibly be spread through the body. After conducting an in vivo study where the hydrogel was placed in the calvaria, the major organs were excised, sectioned, and examined for evidence that hydrogel particulate. None of the sections showed evidence that as the hydrogel degraded, its components were found in any of the major organs, nor did it have a negative impact on their morphology. It is therefore reasonable to conclude that in terms of safety our novel hydrogel is a viable alternative to collagen scaffolds and other common drug delivery vehicles.

As a test of biological function in vivo the hydrogel performed well. Based on our microCT reconstruction and histomorphometric quantifications it did a better job of localizing the bone growth than the collagen scaffold. The collagen scaffold group had a significant amount of bone growth outside the defect, above the normal parietal bone, which we considered to be heterotopic ossification. The hydrogel limited the amount of heterotopic ossification. Based on the histology sections of this bone growth, rhBMP2 release from the hydrogel stimulated healthy ossification. Interestingly, the hydrogel loaded with rhBMP2 seemed to do a better job at closing the defect based on the staining. This is likely due to the fact that it was better able to localize the bone growth.

### Conclusions

The novel click hydrogel represents a promising alternative to the traditional absorbable collagen scaffold typically used in delivery of BMP2. Its ability to crosslink rapidly and without a photo initiator or potentially harmful precursor makes it promising for use in a clinical setting. Its abilities as a tunable vehicle cannot be underestimated. A clear benefit of hydrogel over a typical collagen scaffold is its ability to be injected wherever a needle can fit. In terms of bone fracture treatment, this has immense clinical use in that the hydrogel can be injected to the site of injury to promote healing without invasive surgery.

### Example 3. Semaphorin 3A Delivered by a Rapidly Polymerizing Click Hydrogel Overcomes Impaired Implant Osseointegration in a Rat Type 2 Diabetes Model

Dental implants have enabled the restoration of dentition in the growing aging population, which has resulted in improved health for many individuals. However, poor bone quality associated with diseases like type 2 diabetes mellitus (T2DM) and osteoporosis impacts implant survival. Even though some reports have indicated that the success rate of dental implants in diabetic patients is comparable to non-diabetic patients, current evidence supports the hypothesis that T2DM patients with a poorly or moderately controlled glycemic level have a higher early implant failure rate than well-controlled T2DM patients. Implant success depends on osseointegration.

Sema3A is a known osteoprotective factor that both increases bone formation and inhibits bone resorption. One of the challenges when administering this potent factor as a potential therapeutic is delivering it at an adequate concentration within a sufficient therapeutic window to elicit the desired cellular effects and achieve a favorable outcome in compromised patients. Our strategy for achieving sustained delivery of sema3A was to use a minimally invasive, rapidly polymerizing, click chemistry-based hydrogel. The bio-orthogonal, injectable hydrogel described in this study forms a stable gel in under 90 seconds at 37° C. through a ring-strain promoted Cu-free click reaction between azide functionalized PEG polymers and a DBCO functionalized PEG crosslinker without external photo-initiators and without generating heat. The rapid encapsulation of bioactive molecules into the hydrogel maintains the biologics at the delivery site over an extended time. Ester linkages degrade in vivo and ultimately release the loaded molecules.

In these studies, we employed the Zucker Diabetic Sprague Dawley (ZDSD) rats, a clinically relevant T2DM rodent model. Unlike the prevailing type 2 diabetic rodent models that are either induced by chemical toxin injection (such as streptozotocin or alloxan) or via genetic mutations (leptin or leptin receptor deficiency), ZDSD rats develop T2DM spontaneously, in a manner that closely resembles factors contributing to the manifestation of T2DM in humans. Using this model allows clinicians to better translate findings to benefit the human condition and improve clinical outcomes when managing T2DM patients receiving implants.

Here we report the impact of T2DM on the osseointegration of microtextured titanium implants in the ZDSD rat model and deliver sema3A in two modalities: via direct incorporation into the click hydrogel and via local injection of sema3A over the implant site. In addition, the effect of sustained delivery versus burst release of sema3A is demonstrated in this study. Finally, we assessed the effects of T2DM on the ability of rat calvarial osteoblasts to respond to the Ti surface in vitro.

### Materials and Methods

### Hydro Gel Preparation

The methods for preparing our click hydrogel were described in previous studies. Briefly, the rapidly polymerizing hydrogel was synthesized by mixing two aqueous solutions that undergo in situ chemical crosslinking via copper-free click-based chemistry. Dibenzocyclooctyne-functionalized PEG crosslinker (DBCO-functionalized PEG) was synthesized by Thiol-Michel addition reaction between PEG-dithiol and dibenzocyclooctyne maleimide (DBCO-maleimide). The DBCO-functionalized precursor forms an in-situ crosslinked hydrogel with azide functionalized acylate polymer. To generate a water-soluble non-fouling multivalent azide functionalized polymer, we synthesized PEG-N3 from azide functionalized and non-functionalized PEG methacrylate monomers via reversible addition-fragmentation chain transfer (RAFT) polymerization, which affords tight control of azide functionality. The hydrogel was formed by mixing one part of PEG-N3 (50%; w:v) and two parts of PEG-DBCO (12.5%; w:v) using a dual-syringe dispensing apparatus that dispenses the two solutions simultaneously at a 2:1 ratio.

### Hydrogel Characterization

To assess swelling, the surface areas were analyzed by ImageJ to determine the swelling ratio (qt), normalized to T0. The horizontal degree of swelling was defined by the average swelling ratio (qt)±standard error (SEM), where the surface area of the hydrogel at each time point compared with its initial measurement is divided by the initial surface area. Vertical swelling was assessed by percent weight and volume change under physiological conditions at 37° C. for 1 h, 2 h, 3 h, 8 h, 12 h, 24 h, and 48 h.

To assess gelation, the viscosity-sensitive probe 2-cyano-3-(2,3,6,7-tetrahydro-1H,5H-benzo[ij]quinolizin-9-yl)-2-propenoic acid (CCVJ) solution (Cayman Chemical, Ann Arbor, MI, USA) was loaded into the crosslinker solution and combined with the copolymer to form the click hydrogel, which was visualized by fluorescence microscopy at an emission of 497 nm for 5 minutes. A conservative threshold intensity (>1000 A.U.) was determined by the glycerol standard. Total fluorescence was determined by the number of pixels above the threshold fluorescence intensity.

Rheological measurements (Rheology Testing Services, Chapel Hill, NC) were performed immediately upon hydrogel polymerization at 37° C. A Malvern Kinexus Pro Rheometer equipped with 25 mm roughened parallel plates and a gap height of 400 μm (n=3). Hydrogels were allowed to cure at 37° C. for 5 minutes. The force required to separate the two plates vertically was measured (pull-away speed of 5 mm/s). Force displacement curves were generated to quantify the maximum force required to separate the two plates vertically (tackiness). Amplitude sweep experiments were conducted using increasing oscillatory strain at a constant frequency to determine the hydrogel's linear viscoelastic region (LVER). Frequency sweep experiments were made over a range of oscillation frequencies (0.1-20 Hz) at 37° C. at a constant oscillation amplitude (0.5% strain), a polydimethylsiloxane (PDMS) viscoelastic standard under the same parameters used for the hydrogel samples.

### Proton Nuclear Magnetic Resonance

¹H NMR analysis was performed on a Bruker Avance III 600 MHz spectrometer. 50 to 100 mg of lyophilized copolymer and crosslinker were dissolved in approximately 0.85 mL of deuterated water (D20) or deuterated chloroform (CDCl₃). 1H NMR chemical shifts were at 4.79 ppm for water and 7.26 for chloroform.

Sema3A Release Kinetics and In Vitro Bioactivity of sema3A Released from the Hydrogel

For our release studies, 6 μg recombinant sema3A (R&D Systems) was loaded into the click hydrogel. The gels with or without sema3A were incubated at 37° C. in 300 III, of Dulbecco's modified Eagle medium (DMEM; Mediatech) and 1% penicillin-streptomycin (VWR International). DMEM were collected and replaced on days 1-5. Bio-Dot assay (Bio-Rad Laboratories) was used to quantify hydrogel-released sema3A, according to the manufacturer's instructions. 33 MG63 human osteoblast-like cells (ATCC) were cultured at 10,000 cells/cm2 in a 24-well plate with DMEM with 10% fetal bovine serum (FBS) (Gemini) and 1% penicillin-streptomycin (DMEM FM). Media were replaced with fresh DMEM FM 24 hours after plating and every 48 hours until 80% confluent and then treated with 50% conditioned media with 50% fresh DMEM FM. Twenty-four hours after treatment, DNA content of cell layer lysates was determined. Immunoassays using collected media were performed to measure levels of BMP2, osteopontin (OPN), osteoprotegerin (OPG) (R&D Systems), and osteocalcin (OCN) (Thermo Fisher Scientific).

### Preparation of Titanium Implants and Disks

Grit-blasted, acid-etched 3.5 mm long implants×2.5 mm outer diameter Ti screw implants (SLA, Institut Straumann AG Basel, Switzerland) were machined from grade 4 Ti rod stock as described previously. 34 15-mm diameter SLA Ti disks were punched from 1-mm thick sheets of grade 2 Ti (Institut Straumann AG) manufactured as previously described24 and used for in vitro studies.

### Experiment Design and Surgical Procedures

Thirty 15-week-old male Zucker Diabetic Sprague Dawley rats (ZDSD) and twenty-eight age-matched male Sprague Dawley rats (SD) (Charles River Laboratories, Wilmington, MA, USA) were used.

Twenty-one ZDSD rats developed diabetes, while nine ZDSD rats did not develop diabetes and were used as the genetic control cohort. All animal procedures were approved by the Institutional Animal Care and Use Committee at Virginia Commonwealth University. All experiments were carried out following the National Institutes of Health guide for the care and use of laboratory animals. Animals were housed in a single ventilated, solid-bottomed polysulfone cage in an AALAC-accredited animal facility with indoor housing capable of controlling temperature and humidity within species-appropriate ranges and maintaining a 12 h/12 h light/dark cycle. For all procedures, anesthesia was induced with 5% isoflurane gas inhalation in O2 and maintained with 2.5% isoflurane gas inhalation in O2. Animals were recovered from anesthesia on a water-circulating warming pad before returning to the vivarium. For all surgical procedures, 1 mg/kg of sustained-release Buprenorphine (ZooPharm, Windsor, CO, USA) was administered pre-operatively via subcutaneous injection to provide a minimum of 72 hours of postoperative analgesia.

### Diabetes Induction

At 15 weeks of age, all rats were fed a high-fat diet (D12468, Research Diet, New Brunswick, NJ, USA) to induce diabetes.30,31,35,36 Twenty-one ZDSD rats turned hyperglycemic with nonfasting blood glucose levels higher than 250 mg/dL after 3 weeks. After four weeks, all rats were switched to a standard diet (Purina 5008, LabDiet, St. Louis, MO, USA) and aged for another three weeks before implant surgery. Blood glucose levels were monitored weekly.

### Hydrogel Loading and Implant Placements in Both Hindlimbs

On day 49, diabetic ZDSD rats were randomly assigned to the diabetic empty hydrogel (hydrogel alone) group (Diabetic GEL, N=7 rats, n=14 limbs), the diabetic hydrogel-delivered sema3A group (Diabetic 3A+GEL, N=7 rats, n=14 limbs), or the diabetic locally injected sema3A group (Diabetic i3A+GEL, N=7 rats, n=14 limbs). Twenty-eight SD rats were randomly assigned to Normal GEL (N=9 rats, n=18 limbs), the Normal 3A+GEL group (N=9 rats, n=18 limbs), and the Normal i3A+GEL (N=10 rats, n=20 limbs).

The implant insertion site was produced by sequentially drilling a defect with increasing diameter dental drill bits (Ø1.0 mm, Ø1.6 mm, Ø2.0 mm, and Ø2.2 mm) to a depth of 3.5 mm in the distal metaphysis of the femur after separating the adjacent muscles and periosteum. 6 μg of sema3A (6 μg/limb) was added to 10.66 μL PEG-DBCO crosslinker.37 5.33 μL of PEG-N3 was combined with 16.66 μL of crosslinker mixture by pipetting into the drilled hole simultaneously. After gelation, implants were screwed into the hole immediately, a cover screw applied, and tissues re-approximated and closed with suture and wound clips.

The procedure was repeated on the contralateral limb. Four weeks later, all rats were euthanized by CO2 inhalation. The nine non-diabetic ZDSD rats were aged until 30 weeks old and used as the genetic control group.30 Non-diabetic ZDSD rats were randomly assigned to ZDSD (−) GEL (N=3 rats, n=6 limbs), and the ZDSD (−) 3A+GEL (N=3 rats, n=6 limbs), and ZDSD (−) i3A+GEL (N=3 rats, n=6 limbs). Animals were sacrificed four weeks following implant surgery. i3A+GEL groups received local injections of sema3A (6 μg of 100 μg/mL sema3A in sterile 0.9% NaCl per limb) to both hindlimbs underneath the periosteal layer but above the implant on days 52 and 59. Two diabetic ZDSD rats from the diabetic hydrogel-delivered sema3A group were withdrawn from the study following implantation surgery as they met the humane endpoint.

### Tissue Analysis

Parameters for microCT used an isotropic voxel size of 15.82 μm. Adjacent bone formation was also evaluated qualitatively from microCT images by two independent observers blinded to the treatment groups, with the average of the two observers reported. 0: 0%-25% bone around implants; 1: 25%-50% bone around implants; 2: 50%-75% bone around implants; 3: 75%-100% bone around implants. Gap frequency quantification was done by three independent observers blinded to the groups, with the average being reported. Plastic embedded samples were prepared for histology, imaged by a bright field microscope, and evaluated for peri-implant bone growth and BIC, as described before. Removal torque testing was conducted identically as previously described. A torque vs. radian graph was generated for each implant (not shown).

Tibia bone marrow from diabetic and normal GEL groups was flushed, pulverized, and RNA or protein was extracted. Total RNA was isolated according to the manufacturer's protocols using TriZol reagent (Thermo Fisher Scientific) and the RNeasy Mini kit (Qiagen, Hilden, Germany) to quantify SEMA3A and GAPDH (not shown). Total protein was extracted from 50 mg of bone powder using Minute™ Total Protein Extraction Kit for Bone Tissue (Invent Biotechnologies), following the manufacturer's instructions. Sema3A protein production was measured by rat sema3A ELISA kit (MyBioSource) and normalized to the total protein. Calvarial osteoblasts were isolated using the same protocols as previous studies. Calvarial osteoblasts from the diabetic GEL and SD rats were plated on TCPS or SLA surfaces at 10,000 cells/cm² in DMEM FM for 7d. At 7d, cells were incubated with fresh DMEM FM for 24 hours, and cell-conditioned media were collected for the same immunoassays as above, along with Sema3A (LifeSpan Biosciences) and normalized to DNA.

### Statistical Analysis

Based on previous studies, eight successful implants are required for each group to ensure implant success in systemic compromised conditions with a 30% mean difference, a 20% variance, and a type I error rate of 0.05 using two-tailed one-way ANOVA analysis to maintain 80% power. All statistical analyses are stated in each respective figure legend.

### Results

### Hydrogel Characterization

Force-displacement curves revealed that the hydrogel samples had modest adhesive properties indicated by the negative force. The area under the curve represented the work done by the plate to stretch the hydrogel to its breaking strain and revealed the cohesive properties of the hydrogel. The tackiness of the hydrogel samples was −3.37±0.49 N. The gel remained cohesively intact, and its highly elastic nature allowed it to spring back onto the bottom plate at the point of adhesive failure in approximately half a second (FIG. 9A). The frequency sweep data revealed that the gel was more solid-like than liquid-like in nature and remained polymerized over the oscillatory frequencies measured. The elastic (G′) modulus increased with frequency (0.1-2 Hz) and was larger than the viscous (G″) modulus, indicating a stiffer, more solid-like nature. Additionally, the low (2-8° C.) phase angle supports a firmer, solid-like property, where values between 45 and 90° are more liquid-like. A G′/G″ crossover event was not observed between the storage and loss moduli, demonstrating that the hydrogel remained crosslinked after gelation at the frequencies tested. The lack of significant changes in the G′ and G″ values over the tested time period was an indicator of stable gelation of the hydrogel (FIG. 9B). Oscillatory shear measurements provided the viscosity versus shear rate response of the hydrogel as a measure of shear thinning and ease of injectability. The complex modulus G′ was relatively constant while the complex viscosity (* decreased with angular frequency and increasing shear rate, indicative of shear degradation of the polymer (FIG. 9C). The G′/G″ ratio of elasticity was relatively constant and observed to be 10.28±1.68 at the lowest frequency tested (0.1 Hz) (FIG. 9D). The shear thinning behavior observed verifies the injectability of the hydrogel. Inertia effects were flagged for one sample at two frequencies (15 Hz and 19 Hz), suggesting possible slippage at the plate-sample interface despite using roughened plates and consistent gap force and height (FIG. 9D). The onset of gelation occurred in under 15 seconds with minimal changes in total fluorescent intensity and therefore, viscosity, over 5 minutes. The glycerol standard exhibited low fluorescence intensity, with detectable fluorescence completely abolished after 3 minutes (FIG. 9E). The average horizontal swelling ratio (qt) was determined to be 2.42±0.14 after 48 h, which is indicative of minimal swelling behavior according to the literature, where slightly swelling hydrogels are defined as having swelling ratios below 8.9 and strongly swelling hydrogels have swelling ratios greater than 16.8 and approaching a ratio of 100 (FIG. 9F). The horizontal swelling ratio of our click hydrogel most closely resembled values reported for polymethacrylic acid (hPMAA) click hydrogels. The average vertical swelling after 1 hr was 8.63±0.63% (qt=0.086±0.01) for Method 1 and 8.77±1.75% (qt=0.19±0.19) for Method 2. After 2 hrs, swelling increased to 8.91±0.82% (qt=0.09±0.02) and 21.05±5.26% (qt=0.12±0.04), respectively, and no additional swelling was observed after 48 hrs (FIGS. 1G, and 1H). The ¹H NMR spectra of the copolymer (FIG. 9I) and crosslinker (FIG. 9J) show peaks that correlate with the chemical structure of these hydrogel components.

### ZDSD Rats Spontaneously Developed a Diabetic Bone Phenotype.

Rats were characterized as diabetic when their blood glucose was higher than 250 mg/dL for one week. Table 1 shows that all groups started with similar blood glucose levels lower than 250 mg/dL. On implantation surgery day (day 49) and harvest day (day 77), ZDSD rats had higher blood glucose levels (>250 mg/dL) than normal SD rats. After introducing a high-fat diet, the blood glucose of ZDSD rats gradually increased, and 70% of ZDSD rats turned hyperglycemic after three weeks. The control rats maintained normal glycemic values (FIG. 10A). Diabetic rats had lower body weights than normal rats on the day of implantation (FIG. 10B) and at harvest (FIG. 10C). Trabecular bone was analyzed at the metaphysis of the distal femur (FIG. 10D). Diabetes decreased bone volume/total volume (BV/TV) (FIG. 10E), increased total porosity (FIG. 10F), and inhibited trabecular thickness (FIG. 10G) and number (FIG. 10H).

TABLE 1

| Blood | | | |
|---|---|---|---|
| Glucose (mg/dL) | Day 0 | Day 49 | Day 77 |
| Normal GEL | 114.50 ± 5.63 | 117.22 ± 5.73 | 119.67 ± 8.06 |
| Normal 3A+ GEL | 113.00 ± 7.27 | 113.75 ± 11.37 | 122.22 ± 5.05 |
| Normal i3A+ GEL | 110.80 ± 3.95 | 116.3 ± 2.94 | 115.40 ± 2.48 |
| Diabetic GEL | 122.00 ± 4.07 | 471.71 ± 42.95^{#} | 490.86 ± 35.93^{#} |
| Diabetic 3A+ GEL | 120.20 ± 2.75 | 438.6 ± 53.19^{#} | 529.40 ± 63.71^{#} |
| Diabetic i3A+ GEL | 120.29 ± 5.32 | 475.14 ± 36.34^{#} | 437.43 ± 41.35^{#} |

Sema3A Released from the Hydrogel Maintained its Bioactivity.

Sema3A was gradually released over five days and reached a steady state on day 4 (FIG. 11A). Prior to testing sema3A in vivo, we demonstrated its osteogenic capability in MG63. When MG63 cells were treated with hydrogel-released sema3A, DNA content was reduced (FIG. 11B). Osteocalcin (OCN) (FIG. 11C), BMP2 (FIG. 11D), and osteoprotegerin (OPG) (FIG. 11E) were increased; but no change in osteopontin (OPN) (FIG. 11F) was observed. Conditioned media from empty hydrogels (Empty GEL) were compared to conditioned media from sema3A-loaded hydrogels (Sema3A+GEL). In cells treated with Sema3A+GEL media, DNA was reduced (FIG. 11G); there was no difference in OCN (FIG. 11H); and BMP2, OPN, and OPG were increased (FIG. 11I-K) as compared to cells treated with Empty GEL media. The results from these two experiments demonstrated that sema3A released from the click hydrogel maintained its biologic activity.

### Sema3A Mitigated the Osteopenic Bone Phenotype in T2DM.

Qualitatively, there was less trabecular bone in the diabetic GEL rats than in normal GEL rats (FIG. 12A-F), confirming the quantitative measurements (FIG. 12G-J). In the diabetic rats, sema3A delivered by the hydrogel (labeled as 3A+GEL) increased BV/TV (FIG. 12G), decreased total porosity (FIG. 12H), and increased trabecular thickness (FIG. 12I), but not trabecular number (FIG. 12J), compared to diabetic GEL rats. The effects of diabetes on BV/TV, total porosity, and trabecular thickness (FIG. 12G-I) were eliminated by 3A+GEL but not by sema3A local injection (labeled as i3A+GEL). Both sema3A treatments eliminated the diabetic effect on trabecular number (FIG. 12J). Diabetes did not compromise cortical bone at the metaphysis. There was no change between the normal and diabetic groups in BV/TV, total porosity, and cortical thickness (FIG. 12K-M). However, diabetes decreased the bone perimeter, and only i3A+GEL recovered the difference (FIG. 12N). Diabetes did not affect cortical bone at the mid-diaphysis (not shown), but sema3A delivered by either hydrogel or local injection increased BV/TV (not shown) and decreased total porosity (not shown). Additionally, 3A+GEL increased mean cross-sectional bone perimeter in diabetic rats (not shown). However, 3A+GEL resulted in lower cortical thickness than GEL in the diabetic rats (not shown). There was no difference in sema3A gene expression (not shown) or protein production (not shown) in extracts of tibial bone from normal and diabetic rats treated with GEL.

Sustained Release of sema3A Increased Marrow BIC and BV/TV.

Osseointegration occurred in all groups (FIG. 13A-F). Qualitatively, the attached bone on the titanium implant surfaces was decreased in diabetic rats compared to normal rats (not shown) regardless of sema3A treatment. Quantitatively, total BIC was decreased in diabetic rats and was not modified by sema3A treatments (FIG. 13G). The marrow BIC reduction between normal and diabetic rats remained significant, and 3A+GEL yielded a higher marrow BIC than GEL in the diabetic rats (FIG. 13H). However, i3A+GEL did not restore the marrow BIC to normal. Surprisingly, 3A+GEL decreased the cortical BIC compared to GEL in diabetic animals and exaggerated the difference compared to the normal rats. i3A+GEL ameliorated the difference between normal and diabetic rats in cortical BIC (FIG. 13I). Total adjacent bone formation was not compromised by diabetes, indicated by no significance observed in total BV/TV (FIG. 13J). The total adjacent bone was separated into trabecular and cortical bone volume fractions.

Diabetes dramatically reduced trabecular bone formation, which 3A+GEL increased. Both sema3A treatments eliminated the diabetic effect on trabecular bone volume (FIG. 13K). Cortical bone formation was not affected by diabetes or sema3A treatment (FIG. 13L). By observing three-dimensional microCT images, diabetes qualitatively reduced adjacent bone formation, and both sema3A treatments ameliorated the bone reduction (FIG. 13M).

### Sema3A Increased Total BIC Regardless of Delivery Methods.

Qualitatively, histology showed less adjacent bone in the T2DM rats than in normal rats (FIG. 14A-F). Total and marrow BIC were dramatically reduced in untreated T2DM animals, and sema3A treatment eliminated the diabetic effect (FIG. 14G-H). Cortical BIC was not affected by diabetes, and diabetic 3A+GEL had lower cortical BIC compared to other diabetic groups (FIG. 141). Cortical thickness was not affected by diabetes itself, but i3A+GEL treatment did amplify the difference between normal and diabetic rats (FIG. 14J). Total adjacent bone formation was not affected by diabetes but increased in the 3A+GEL group compared to the diabetic GEL group (FIG. 14K). Gaps were observed by microCT and histology between the cortical bone and implant surfaces in some samples. Gap frequency was greatest for diabetic rats compared to normal rats, and diabetic rats that received sema3A in the metabolically active bone marrow space had the highest gap frequency of all groups (not shown).

### Sema3A Enhanced the Bone Mechanical Properties in T2DM.

Diabetes reduced the maximum torque and torsional stiffness compared to the normal GEL (FIGS. 15A and B). Both sema3A treatments mitigated the diabetic effect on maximum torque (FIG. 15C) and torsional stiffness (FIG. 15D). Moreover, sema3A increased the torsional stiffness compared to the diabetic GEL regardless of delivery method (FIG. 15D).

T2DM Cells do not Produce More sema3A on SLA Surfaces.

Calvarial osteoblasts from normal rats produced more sema3A on SLA than TCPS (FIG. 16A), but this was not observed in cultures of osteoblasts from diabetic rats (FIG. 16B). Diabetic osteoblast cultures had less DNA on SLA than TCPS. Sema3A treatment did not affect the DNA amount on TCPS but decreased it on SLA (FIG. 16C). Diabetic osteoblasts produced more OCN on SLA than on TCPS, and sema3A further enhanced the OCN production on SLA (FIG. 16D). Similar data were observed with BMP-2 (FIG. 16E) and OPG production (FIG. 16F). OPN production by diabetic osteoblasts was higher on SLA than TCPS and was not affected by sema3A treatment (FIG. 16G). No difference in VEGF-165 production was observed regardless of surface or treatment (FIG. 16H).

### SD and ZDSD (−) are Optimal Non-Diabetic Controls.

30% of the ZDSD rats that did not turn diabetic were used as genetic controls (ZDSD (−)). Normal Sprague Dawley rats (SD) and ZDSD (−) had no difference in BV/TV, total porosity, trabecular thickness, and number (not shown). Diabetes caused trabecular bone reduction compared to both controls (not shown). No difference was observed in the cortical bone phenotype of SD and ZDSD (−) (not shown). Diabetic rats increased cortical BV/TV compared to the ZDSD (−) (not shown) and decreased mean cross-sectional bone perimeter compared to both controls (not shown). ZDSD (−) exhibited similar total BIC and marrow BIC to the SD (not shown). In contrast, they had lower cortical BIC than the SD (not shown). Diabetic rats had comparable levels of all BIC compared to ZDSD (−) (not shown). However, when BIC was analyzed by histology (not shown), diabetes reduced all BIC compared to both controls (not shown). There was no difference between the control groups regarding total adjacent bone formation, but diabetic rats had less BV/TV than the genetic control (not shown).

There was no difference in cortical thickness among the three groups (not shown). There was no significant difference between the SD control rats and the genetic control rats regarding bone phenotype and osseointegration. To validate that the ZDSD genetic control can be used as a non-diabetic control in terms of sema3A treatment, we performed microCT and histologic assessment comparing the genetic control and T2DM (not shown).

### Discussion

In this study, we proved the use of our injectable click hydrogel as an effective delivery platform for biologics, specifically sema3A, as a therapeutic to treat impaired osseointegration of implants in compromised patient populations, such as T2DM. Characterization of the hydrogel revealed attractive mechanical and physicochemical properties, biocompatibility, and rapid in situ polymerization without using toxic photo-initiators or organic solvents. These properties demonstrated the broad applicability of our click hydrogel and enabled its potential use in various regenerative and drug delivery applications, especially in challenging implantation environments.

The rapid in situ polymerization of the hydrogel minimizes material migration during gelation while enhancing the mechanical properties such as stiffness without compromising flow properties necessary for injectability. Compared with most conventional hydrogels described in the literature, our click hydrogel showed substantially less swelling under physiological conditions, especially vertical swelling when confined to the constraints of a container. Reduced swelling in an aqueous environment correlates with improved mechanical properties, reduced slippage from the application site due to minimal temporal changes in shape, and fewer instances of local nerve compression. Additionally, the complex viscosity and G′/G″ ratio of elasticity observed with our hydrogel were similar to the viscoelastic properties reported for soft tissues likely contributing to the cytocompatibility of the hydrogel, where its similar viscoelasticity is not expected to alter the local biological and mechanical cues produced in vivo.

Sema3A is a well-known osteoprotective factor that works during the bone remodeling phase. The sustained release of sema3A increases the likelihood of maintaining the protein at the needed location in the appropriate time frame to maximize the effect of sema3A on bone remodeling compared to a burst release strategy. The delivery via this hydrogel platform resulted in increased BIC and adjacent bone formation in the metabolically active bone marrow. Sema3A increased the bone volume fraction and improved the bone quality, indicated by enhanced torsional stiffness, by increasing the ability of the bone to recover under stress. This is important for patients with higher fracture risks due to poor bone quality. The two burst releases of sema3A by injection to the cortical bone around the implants enhanced the cortical BIC, suggesting that a strategy of sustained release of sema3A to both the marrow space and the surrounding cortical bone is critical to maximizing osseointegration.

The effect of sema3A on osseointegration can be attributed to increasing trabecular bone and improving the cellular response to titanium surfaces. We observed that sema3A released by the hydrogel promoted trabecular bone growth and eliminated the differences between normal and diabetic animals. Diabetes has been shown to impair cell functions and induce apoptosis. Chronic hyperglycemia alters the response of osteoblasts to parathyroid hormone, leading to decreased differentiation. Our results showed that T2DM osteoblasts still respond to SLA surfaces, indicated by the increase of osteogenic markers. However, these cells did not produce more sema3A. The inability of T2DM osteoblasts to produce more sema3A on the SLA surfaces may be the cause of impaired osseointegration in diabetic rats. The basal level of sema3A in diabetic osteoblasts is higher than in normal rat osteoblasts.

We propose that diabetic rats produced more sema3A to compensate for diabetes but could not respond appropriately to the surface signals of the implants in vivo. Adding sema3A enhanced the surface-induced osteoblast differentiation, perhaps explaining the effect of sema3A in vivo. Given that nerve-derived sema3A regulates bone remodeling by modulating sensory nerve projections into bone, our sema3A treatment may improve the osseointegration by compensating for the lack of nerve signals or promoting nerve growth into bones.

### Conclusions

Our study has identified that trabecular bone formation and osseointegration were decreased in a clinically translatable type 2 diabetic rat model. Furthermore, both the sustainable and burst release of sema3A at the implant site was shown to restore diabetic-impaired osseointegration and halt the progression of the trabecular bone resorption. These in vivo results were supported by in vitro sema3A treatment studies showing that sema3A may increase the peri-implant bone growth by improving the osteoblastic differentiation on SLA disks.

We are the first to demonstrate the sustained release of sema3A to the implant site using a rapidly polymerizing click hydrogel as a drug delivery system. This strategy allowed rapid encapsulation of sema3A at the local delivery site while retaining the bioactivity of the protein. This method has important implications for applications in regenerative medicine. Importantly, we demonstrated the therapeutic efficacy of sema3A on bone healing and regeneration in a clinically translatable T2DM rodent model.

### Example 4. Osseointegration of Titanium Implants in a Botox-Induced Muscle Paralysis Rat Model is Sensitive to Surface Topography and Semaphorin 3A Treatment

The success rate of dental, spinal, or orthopedic implants has always been challenging for compromised patients, such as the elderly, smokers, diabetics, and osteoporosis. To increase implant success, research on implants with modifications such as complex micro-/nano-topography, wettability, and altered surface chemistry has been ongoing for many years. Accordingly, we used the botox injection model to evaluate the effectiveness of biomimetic surface topography in the absence of mechanical loading and to assess if the local delivery of the nerve-derived factor sema3A would improve osseointegration to the level found in healthy animals. The study determined whether sema3A treatment is sufficient to stimulate osseointegration with a smooth Ti implant to the levels observed when using a Ti surface with a multiscale biomimetic topography. We then assessed if the addition of sema3A is able to enhance the osseointegration of a Ti implant with a biomimetic surface in the botox-compromised model.

### Materials and Methods

### Implant Manufacturing

Titanium Implants machined from grade 4 titanium rods to be 2.5 mm in diameter, 3.5 mmin length, and 0.8 mmin pitch were customized to fit in a rat femur by Institut Straumann AG (Basel, Switzerland). The machined implants were designated “pre-treatment” (PT). The PT implants were blasted with 250-500_m Al₂OH₃ grit and acid-etched in a mixture of HCl and H2504, resulting in a complex microrough topography (SLA), and then processed under nitrogen and stored in 0.9% sterile saline, resulting in a hydrophilic surface that had nanoscale features hydrophilic (modSLA). PT and modSLA implants were sterilized using irradiation. In order to obtain hydrophobic implants that had both the SLA microroughness and the added nanoscale features found on the modSLA surfaces, the modSLA implants were removed from the sterile saline package in a biological safety cabinet under sterile conditions and aged for at least 1 month to generate the SLAnano surfaces, which were repackaged in aluminum foil. The physical and chemical properties of the PT and SLAnano surfaces have been described in detail.

In the present study, our goal was to focus on surface topography without the confounding variable of wettability, and the PT and SLAnano surfaces are both hydrophobic. Thus, the present experimental design enabled us to focus on the contribution of biomimetic multiscale topography on osseointegration in this mechanical unloading model and to assess the contribution of sema3A to the process.

### Hydrogel Preparation

We used a copper-free click hydrogel as the sema3A delivery vehicle. We successfully used the click hydrogels to deliver biologics with no evidence of toxicity. Swelling of the hydrogel after administration is minimal, and following an initial burst release, the payload is released at a steady rate, with degradation occurring over a 14-day period. The techniques used to prepare the hydrogel are as follows: the copper-free click-based chemistry was used to combine two aqueous solutions that underwent in situ chemical crosslinking to create the quickly polymerizing hydrogel. A thiol-Michel addition reaction involving PEG-dithiol and dibenzocyclooctyne maleimide (DBCO-maleimide) was used to create a poly-ethylene glycol (PEG) crosslinker that has been functionalized with dibenzocyclooctyne (DBCO). When combined with an azide-functionalized acylate polymer, the DBCO-functionalized precursor created an in situ crosslinked hydrogel. Reversible addition-fragmentation chain transfer (RAFT) polymerization, which allows for the precise control of azide functionality, was used to create PEG-N3 from azide functionalized and non-functionalized PEG methacrylate monomers to produce a water-soluble, non-fouling multivalent azide functionalized polymer. The components were synthesized at a commercial facility under Good Laboratory Practice controls (Syngene International Limited, Bangalore, India) according to our requirements and shipped lyophilized to our laboratory. Before use, the components were stored at −80° C. after being reconstituted in sterile 1×PBS (ThermoFisher Scientific, Waltham, MA, USA). The hydrogels were formed by combining PEG-N3 (50%; w:v) and PEG-DBCO (12.5%; w:v) at a 1:2 (v/v) ratio.

### Animals and Surgical Procedures

The Institutional Animal Care and Use Committee at Virginia Commonwealth University approved all the animal procedures. The National Institutes of Health's guide for the care and use of laboratory animals was followed in all experiments. Animals were kept in an AALAC-approved animal facility in indoor housing with a 12 h/12 h light/dark cycle and individually ventilated, solid-bottomed polysulfone cages that allow for temperature and humidity adjustment within ranges appropriate for the animals.

For all animal procedures, 5% isoflurane gas with O₂ was used to induce anesthesia and kept at 2.5% after. Animals recovered consciousness on a water-circulating warming pad before returning to the vivarium. For surgical procedures, 1 mg/kg of sustained-release buprenorphine (ZooPharm, Windsor, CO, USA) was administered pre-operatively and subcutaneously to provide a minimum of 72 h of postoperative analgesia.

### Therapeutic Effect of Sema3a on the Botox-Induced Compromised Bone Phenotype

In total, 29 male Sprague Dawley rats (SD) weighing 300-325 g (Charles River Laboratories, Wilmington, MA, USA) were randomly divided into 4 groups: control (veh, n=4), control with sema3A injections (veh+sema3A, n=8), botox injections (BTX, n=8), and botox injections with sema3A injections (BTX+sema3A, n=9). One extra rat was prepared and randomly assigned to the BTX+sema3A group. Botulinum toxin type A (onabotulinumtoxinA; BOTOX®, Allergan, Inc. Irvine, CA, USA [botox]) was dissolved in 0.9% saline (10 units/mL). On day 1 and day 25, for the BTX and BTX+sema3A groups, the right hindlimbs were injected intramuscularly with a total of 8 units of botox distributed as 2 units into the following locations: paraspinal muscles, quadriceps, the hamstrings, and the calf muscles. The contralateral legs were the internal controls (FIG. 1a). On day 21 and day 28, recombinant human sema3A was reconstituted in 0.9% sterile saline (100 μg/mL, R&D Systems, Minneapolis, MN, USA), and 6 μg of sema3A (100 μg/mL in 60 μL) were injected into the periosteal layer of the distal end of the third trochanter on the right femurs for veh+sema3A and BTX+sema3A groups, and the same amount of 0.9% sterile saline was injected to the rest of the groups (FIG. 1a). On day 38, the rats were humanely euthanized by CO2 inhalation and cervical dislocation. Femurs were harvested in 1×PBS and further evaluated by microCT and 3-point bending fracture analysis, which is described elsewhere herein.

We opted to use recombinant human sema3A instead of recombinant rat sema3A for two reasons. We knew that human sema3A could enhance surface-mediated osteoblast differentiation of human MSCs in vitro, as well as the production of factors associated with osteogenesis. Moreover, human sema3A restored the osseointegration of Ti implants in a type 2 diabetic rat model to normal levels, demonstrating that it was bioactive in vivo. Therefore, the concentrations of the same human recombinant sema3A were adopted for this study.

### Effect of Surface Topography on Response to Sema3A

In total, 49 male SD rats weighing 300-325 g (Charles River) were randomly divided into 6 groups: control rats with PT implants (Control+PT, n=8), control rats with SLAnano implants (Control+SLAnano, n=8), botox-injected rats with PT implants (BTX+PT, n=8), botox-injected rats with PT implants and sema3A injections (BTX+PT+sema3A, n=8), botox-injected rats with SLAnano implants (BTX+SLAnano, n=8), and botox-injected rats with SLAnano implants and sema3A injections (BTX+SLAnano+sema3A, n=8). One animal from the BTX+SLAnano group was withdrawn from the study as it met the humane endpoint; thus, BTX+SLAnano had n=7 for the subsequent tissue analysis. On day 1 and day 28, the same dose of botox was injected into the same muscle groups to BTX+PT, BTX+PT+sema3A, BTX+SLAnano, and BTX+SLAnano+sema3A right hindlimbs. On day 21, all rats were prepared for implant insertion and hydrogel loading surgeries by shaving and cleaning the right hindlimbs with 70% ethanol and 2% chlorhexidine. The implant insertion sites were produced by sequentially drilling a defect with increasing diameter dental drill bits (Ø1.0 mm, Ø1.6 mm, Ø2.0 mm, and Ø2.2 mm) to a depth of 3.5 mm in the distal metaphysis of the femur after separating the adjacent muscles and periosteum (FIG. 17A).

Recombinant human sema3A (R&D Systems) was reconstituted with the PEG-crosslinker solution. The hydrogels were formed by combining 5.33 μL of PEG-N3 10.66 μL of PEG-DBCO with or without 6 μg of sema3A to the designated groups separate pipettors to pipette two components into the holes simultaneously. Threaded PT implants or SLA implants were inserted into the holes after gelation. Cover screws were added to cap the implants. Then, the hydrogels were delivered on top of the implants again with or without sema3A with the same (FIG. 17B). Rats were recovered from anesthesia on a water-circulating warming pad and weighed weekly. On day 49, all rats were humanely euthanized, and femurs were harvested in 1×PBS for further analysis.

### Tissue Analysis

### Micro-Computed Tomography

Femurs were isolated and prepared for microCT scanning (SkyScan 1173, Bruker, Kontich, Belgium) within 24 h of harvest without fixation to evaluate the bone phenotype and peri-implant bone growth. For the first study, to evaluate the effect of sema3A on bone morphology, both distal and proximal ends of the femurs were scanned at a resolution of 1120×1120 pixels (isotropic voxel size of 15.82 μm) using a 1.0 mm aluminum filter, at an exposure of 250 ms, with scanning energies of 85 kV and 94 μA. A standard Feldkamp reconstruction was conducted by NRecon Software (Bruker) with a beam hardening correction of 20%, and no smoothing was applied. The quantitative trabecular morphometric parameters, including bone volume/total volume (BV/TV), trabecular number, trabecular thickness, and total porosity, were evaluated for the first animal study. The quantitative cortical morphometric parameters were determined, including BV/TV, the total porosity, and cortical thickness.

For the second animal study, bone-to-implant contact (BIC) was evaluated for each implant by scanning the metaphysis of the distal femurs using a 0.25 mm brass filter, at an exposure of 420 ms, with scanning energies of 120 kV and 66 μA. After reconstruction, total BIC, BIC in the marrow space, and cortical bone BIC were determined using previously described methods [Deng, J.; Cohen, D. J.; Redden, J.; McClure, M. J.; Boyan, B. D.; Schwartz, Z. Differential Effects of Neurectomy and Botox-Induced Muscle Paralysis on Bone Phenotype and Titanium Implant Osseointegration. Bone 2021, 153, 116145].

### Mechanical Testing

In the first experiment, a 3-point bending study was performed using a BOSE Electro-Force 3200 Series III axis (TA Instruments, New Castle, DE, USA). Bones were positioned so that the femur's sema3A injection site was in the middle of two support struts that were facing up. The load cell was attached using a triangular prism-pointed testing mount. An axial compressive displacement rate of 0.1 mm per second was used to achieve axial displacement until failure.

In the second experiment, mechanical torque to failure was used to determine the overall implant mechanical integrity using a Bose ElectroForce 3200 Series III Axial-Torsion mechanical testing system equipped with a 445 N/5.7 N m load/torque transducer. Before loading the samples, the load cell was zeroed, and a 0.1 Hz filter was used to cancel the background noise. Femurs were mounted on customized polylactic acid holders with polyurethane adhesive. The implant mount was customized to fit the implant after removing the cover screws on the top. The sides of the bone holder were then clamped between two flat specimen clamps once the implant had been firmly secured to the mount and was perpendicular to the axis of rotation. The implant was subsequently removed from the surrounding bone by rotating the femurs at 0.1°/s while rising at a pace of 0.8 mm/360° simultaneously.

After zeroing the initial load and displacement or initial torque and rotation radian, the mechanical examination was done by creating load vs. displacement graphs for 3-point bending analyses and torque vs. radian graphs for torsional analyses. SLM-Shape Language (Modeling version 1.14, MATLAB, MathWorks, Natick, MA, USA) fitted the curve to a bilinear model to separate the linear region from the toe region to eliminate the initial gap potentially between the mount and the implant. The curve was then evaluated for the maximum load at failure (N), stiffness (N m), and toughness at failure (millijoules) and normalized to the cross-sectional area calculated in the microCT analysis for each leg 3-point bending analyses. For the implant osseointegration analysis, it was possible to compute the torque at failure (Nm), torsional stiffness (linear region slope, N m/radians), and torsional energy (area under the curve, millijoules) from the torque vs. degree graphs. The information is displayed as the treatment (right leg) over the control (left leg).

### Statistical Analysis

A power analysis was performed using an alpha of 0.05 and a power of 80% (delta=5, sigma=3, m=1), which revealed that a minimum of n=7 per group was required for the study to be statistically significant. An in vivo assessment was done between contralateral legs and treatment legs by Wilcoxon matched-pairs signed rank test (α=0.05) represented by an asterisk (*) using GraphPad Prism (GraphPad, La Jolla, CA, USA), and a one-way analysis of variance to compare between groups with Tukey's post hoc test using JMP statistical software (SAS Institute Inc., Cary, North Carolina). A two-way ANOVA was used to compare differences among groups with two independent variances for removal of the torque mechanical test analysis using GraphPad Prism.

### Results

### Botox Compromised the Trabecular and Cortical Bone Phenotype at the Distal Metaphysis of the Femurs

The trabecular bone and cortical bone phenotype at the distal ends of the femur near the implant insertion site were analyzed by microCT (FIG. 18A), and the representative images are shown in FIG. 18B-I. The development of a compromised bone phenotype induced by botox injections was demonstrated qualitatively by reduced the trabecular bone formation (FIG. 18H) compared to both vehicle groups (FIG. 18B, F) and its contralateral leg (FIG. 18D). This was further confirmed quantitatively, including a lower BV/TV (FIG. 18J), higher total porosity (FIG. 18K), and lower trabecular thickness and number (FIG. 18L, M). The addition of sema3A did not have any significant effect on increasing the trabecular bone formation in both healthy rats (veh+sema3A, FIG. 18J-M) and botoxinjected rats (BTX+sema3A, FIG. 18J-M). The cortical bone was also affected by a botox injection. Cortical BV/TV was reduced (FIG. 18N), the cortical bone total porosity was increased (FIG. 18O), and the cortical thickness was reduced (FIG. 18P), indicating that botox decreased the cortical bone formation. Additionally, sema3A did not affect the cortical bone formation in healthy or botox-injected rats at the distal metaphysis.

### Sema3A Burst Release had a Therapeutic Effect on the Botox-Compromised Cortical Bone at its Injection Sites

The cortical bone phenotype was evaluated at the sema3A injected sites at the distal side of the third trochanter and at the mid-diaphysis (FIG. 19A) and was compared to the contralateral legs. The difference caused by a botox injection on the cortical bones was hard to distinguish in the qualitative images (FIG. 19B-I). However, microCT showed that botox reduced BV/TV at the trochanter, and sema3A injections at that site had no effect (FIG. 19J). Botox increased the total porosity (FIG. 19K) and decreased the cortical thickness (FIG. 19L). The Sema3A injections restored the total porosity to normal levels in the botox-treated rats but had no effect on the cortical thickness. Similarly, botox injections reduced BV/TV (FIG. 19M), increased the total porosity (FIG. 19N), and decreased the cortical thickness (FIG. 19O) at the mid diaphysis in comparison to the contralateral legs, and the injection of sema3A had no effect. Overall, botox injections affected the whole bone phenotype by compromising both the trabecular bone and cortical bone formation, and the effect of sema3A on rescuing the compromised bone phenotype was localized and specific to its injection sites.

Biomimetic Surface Topography Improved Osseointegration, and this was Enhanced by Sema3A Treatment

PT and SLAnano implants were inserted into the metaphysis of the distal femurs as described in the methods. The representative images are shown in FIG. 20A-C for SLAnano implants. Botox injections caused a reduction in trabecular bone in the bone marrow compartment, regardless of whether the rats were treated with sema3A (FIG. 20-A-C). Botox reduced the total BIC and cortical BIC compared to the vehicle control groups (FIG. 20A, D, F), but botox injections did not affect BIC in the bone marrow compartment (FIG. 20E). Even though it was not significantly different, marrow BIC was 18% less in the BTX group than in the control group. The addition of sema3A to the BTX group had a mean of 25% for marrow BIC for SLAnano, which was higher (not statistically significant) than both marrow BIC in the botox (18.22%) and control group (22.95%) (not shown). The addition of sema3A eliminated the difference in total BIC between the healthy and botox-injected rats, mainly due to significantly higher cortical BIC after a sema3A treatment in the BTX group (FIG. 20D, F).

PT implants had qualitatively fewer bone trabeculae associate (with them than were present around SLAnano implants (compare FIG. 20A, G). The PT implants did not alter the botox-compromised bone phenotype (FIG. 20H, I). The total BIC for PT implants in botox-treated animals was significantly lower than in the control groups, and the BIC mainly contributed to the decrease in the bone marrow space. This was different from SLAnano implants, in which the decreased total BIC was mainly contributed by lower cortical BIC in the botox-treated rats. Sema3A did not show any effects on improving BIC for PT implants, but there was a therapeutic effect on improving BIC for SLAnano implants under botox-compromised conditions (FIG. 20 D,F).

Ti Surfaces with a Multiscale Biomimetic Topography Improve Osseointegration for Mechanical Unloading Situations Regardless of Sema3A Treatment

Mechanical analysis of hindlimbs by three-point bending (FIG. 21A) showed no differences between vehicle and botox treatment with or without sema3A treatment for the maximum load (FIG. 21B), stiffness (FIG. 21C), and toughness (FIG. 21D). Mechanical torque to failure was used to quantify the material properties of the newly formed bone around PT and SLAnano implants. In healthy rats, SLAnano implants robustly increased the maximum load (FIG. 21E), torsional stiffness (elastic modulus) (FIG. 21F), and yield point (FIG. 21G), which was consistent with the higher amount of trabecular bone observed in representative microCT images (FIG. 19A, G). Compared to PT, the use of SLAnano in BTX-compromised rats increased the integrated bone mechanical properties more than three-fold (not shown). Botox injections reduced the maximum load, torsional stiffness, and yield point for rough titanium implants regardless of sema3A treatment (56% reduction) (FIG. 21E-G). At the same time, there was no difference in the mechanical properties of bone integrated into PT implants when comparing botox-injected rats with healthy rats (FIG. 21E-G). Additionally, sema3A did not affect the mechanical properties of bone attached to either type of implant. Overall, our data showed that titanium implants with a biomimetic surface topography demonstrated the clinical advantages of increasing osseointegration for mechanically unloaded situations compared to smooth titanium implants. The addition of sema3A increased the amount of bone attached to the implants while not affecting the mechanical properties.

### Discussion

In this study, we did not observe any botox-related toxicity issues in terms of disrupting normal eating, high-stress levels, or other concerns. The results of the present study showed that botox treatment reduced the mechanical stability of transcortical Ti implants. The effect was greatest for implants that lacked a biomimetic surface topography. Our results also show that treatment with sema3A via injection did not mitigate the effects of mechanical unloading resulting from botox injection. However, if sema3A was delivered to the treatment site in a biodegradable Cu-free click hydrogel, it was able to mitigate the impact of botox. Interestingly, this ability to overcome the negative impact of botox was limited to sites receiving implants with biomimetic surface topography.

Pathologies, where the muscle function is chronically disrupted, have been proven to affect skeletal health. In conditions such as bed rest and spinal cord injury, bone loss is rapid and acute, ranging from 5% to 25%, depending on the skeletal sites and injury severity. Rodent models representing these clinically mechanically unloaded or muscle disuse conditions, including tail suspension, cast immobilization, intramuscular botox injections, and tendon resection, exhibit bone loss, which might potentially jeopardize bone regeneration.

Our results showed that botox injections dramatically decreased trabecular and cortical bone in femurs at three different locations: the distal metaphysis, the mid-shaft, and the proximal side. The muscle paralysis induced by botox injections compromised the whole bone phenotype, whereas the contralateral legs were unaffected.

Osseointegration is a complex biological event consisting of stem cell recruitment, primary bone formation, bone remodeling, and mature bone formation. Improvements in osseointegration can be approached by improving net bone formation during primary bone formation or by balancing bone formation and bone resorption during the remodeling phase, both of which are interrupted by diseases such as osteoporosis and diabetes. In the current study, a nerve-derived factor, sema3A, was used to evaluate its therapeutic potential in improving bone formation. Sema3A has been shown to increase osteoblastic differentiation, inhibit osteoclast resorption in vitro, and improve bone formation in animal models, including osteoporotic rabbits and mice and diabetic rats. Our data indicate that sema3A successfully improves botox-induced cortical bone loss to a similar level as the healthy rats with only two burst releases to the periosteum of the third trochanter.

Our data also showed that the effect of sema3A was extremely localized. This may have been due to the limited residency of sema3A after the injection of the periosteum. We did not test this clinically important question in the present study. If the effect of sema3A is limited to the injection site, it can be used locally in areas of regeneration without affecting the bone distal to the area of regeneration.

We used a rapidly polymerizing Cu-free click chemistry hydrogel to achieve local sema3A delivery to the implant insertion site. The roughness produced by the grit-blasting and acid-etching processes resulted in craters varying from 30 to 100 μm, overlaid with pits in the range of 1 to 3 μm. SLAnano has additional mesoscale and nanoscale features. This complex multi-scale topography contributed to better bone development and osseointegration by mimicking the natural structure of osteoclast resorption pits on the normal bone surface. Here, we investigated the contributions of surface topography for improving osseointegration in this compromised model and showed that implants with a microscale/mesoscale/nanoscale structure significantly improved regenerated bone quality by improving the maximum load the bone can bear before failure, increasing the recovery ability of the bones at certain loads, and the higher endurance of loads before permanent damage occurs. Our data indicate that biomimetic surface topography contributes to the success of additive approaches, potentially by providing an osteogenic microenvironment that can be further enhanced pharmacologically. Sema3A treatment further enhanced BIC on multiscale biomimetic surfaces but not on smooth surfaces, consistent with in vitro observations. Sema3A increased the production of osteoprotegerin by MSCs cultured on Ti surfaces with a biomimetic topography. Sema3A is a coupling factor that can increase bone formation and decrease bone resorption and osteoprotegerin is a decoy factor that can inhibit osteoclast differentiation. Sema3A increased BIC by increasing bone formation or decreasing bone resorption, achieving net bone formation.

The present data also show that the osteogenic factors generated by cells on biomimetic multiscale topographies work in concert with exogenous sema3A, whereas cells on smooth surfaces either do not produce these factors or produce them at concentrations that are not sufficient for a synergistic effect, especially in compromised bone conditions. The use of botox in this study created a mechanically unloaded situation that mimics clinical conditions, such as patients with neuromuscular injuries and spinal cord injuries or are recovering from prolonged bed rest or long-term use of wheelchairs, as well as patients who have experienced microgravity. Our findings showed that data impaired osseointegration was improved by surface modifications to mimic the natural bone environment in combination with sema3A, although sema3A did not show the advantages of improving either the whole bone mechanical properties or mechanical properties of regenerated bone around implants at the periods we checked, even though our data showed that sema3A increased bone formation and BIC. The results of the present study indicate a therapeutic method to improve osseointegration in patients with compromised bone regeneration.

### Conclusions

The biomimetic concept of providing surface multiscale topography to resemble the natural bone structure is a promising tool for enhancing osseointegration in compromised bone-like disuse conditions, especially when surface modifications are combined with local factors produced by surface-cultured osteoblastic lineage cells. Titanium implant surfaces with a multiscale micro/nano texture exhibited the advantage of increasing the mechanical properties of integrated bone in both healthy and botox-compromised rats. Moreover, with the addition of sema3A, the deleterious effect of botox on osseointegration was restored to healthy levels.

### Example 5. Comparative Studies

The hydrogel disclosed herein (QuickGel) and DuraSeal® dural sealant were incubated at 37° C. in physiological buffer (PBS) for 48 hours. The swelling ability was measured by the change in surface area, mass, and volume of the hydrogel between steady-state and upon suspension in a physiological buffer. The results are presented in FIG. 22. As can be seen, QuickGel and DuraSeal both exhibit an initial increase in volume, but swelling of QuickGel stabilizes at that initial level whereas DuraSeal continues to swell over the time course of the study. This is an important difference clinically when hydrogels are used in small spaces and confined spaces. The limited swelling observed with QuickGel is also important when used to seal dural tears or suture sites because there is less damage to the wound during healing. Another benefit of the QuickGel formulation is the lack of degradation products that cause tissue toxicity.

A 1 or 3 mm diameter defect was introduced in pig dura. The abilities of the presently disclosed hydrogel (QuickGel), DuraSeal™ and Adherus® to seal the leak were tested. A commercial device was used to deliver the FDA approved products DuraSeal™ and Adherus®. A laboratory device was used to deliver the disclosed hydrogel. The results are depicted in FIG. 23. As can be seen, QuickGel was as effective as either commercial product for sealing the 1 mm diameter defect. QuickGel also effectively sealed the 3 mm diameter defect. These findings demonstrate that the sealing property provided by QuickGel is achieved without the swelling typical of currently marketed hydrogels, and suggest that the polymerization mechanism provided by the Cu++-free click chemistry occurs throughout the gel and stabilizes it in situ. QuickGel is delivered to the site as a liquid, with only minimal if any polymerization during delivery; polymerization occurs in situ at body temperature, providing greater adhesion to the underlying tissue.

While the invention has been described in terms of its preferred embodiments, those skilled in the art will recognize that the invention can be practiced with modification within the spirit and scope of the appended claims. Accordingly, the present invention should not be limited to the embodiments as described above but should further include all modifications and equivalents thereof within the spirit and scope of the description provided herein.

We claim:

1. A method of treating a subject in need thereof, comprising
providing an aqueous formulation at a selected location in or on the subject, wherein the aqueous formulation comprises
linkers having the formula 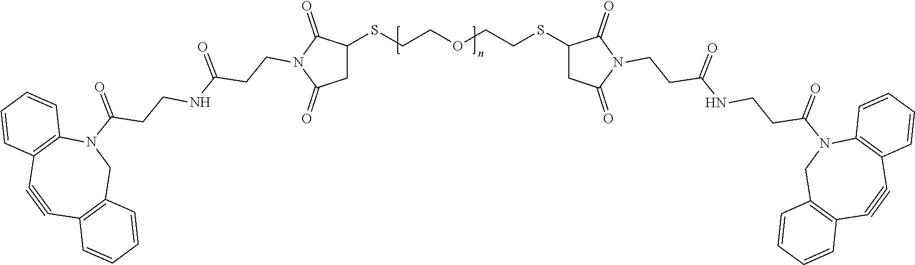
wherein n ranges from 10 to 100, inclusive; and
water-soluble azide functionalized acylate polymers have the general formula 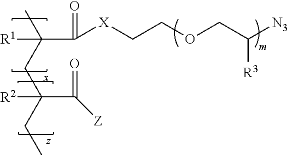
where
R¹ and R² are each independently hydrogen or a C₁ to C6 straight chain, branched chain or cyclic hydrocarbon;
R³ is hydrogen or methyl;
X is —O— or NR⁵— where R⁵ is hydrogen or C₁ to C6 straight chain, branched chain or cyclic hydrocarbon hydrocarbon;
Z is —OR⁶ or NR⁵R⁶ where
R⁵ is hydrogen or C₁ to C6 straight chain, branched chain or cyclic hydrocarbon, and
R⁶ is hydrogen, a C₁ to C6 straight chain, branched chain or cyclic hydrocarbon, or a polyethylene glycol chain of two to ten ethylene glycol units;
m is an integer greater than or equal to 1;
x is an integer greater than or equal to 1; and
z is zero or an integer greater than or equal to 1.

2. The method of claim 1, wherein the selected location is a bone fracture, a wound or a tear or hole in dura matter.

3. The method of claim 1, wherein the aqueous formulation further comprises at least one therapeutic agent.

4. The method of claim 3, wherein the at least one therapeutic agent is a protein, a nucleic acid, an antibiotic, a polyphenol, a vitamin or a mineral.

5. The method of claim 3, wherein the at least one therapeutic agent is semaphorin 3A.

6. The method of claim 3, wherein the at least one therapeutic agent is vancomycin or tobramycin.

7. The method of claim 3, wherein the at least one therapeutic agent is resveratrol.

8. The method of claim 1, wherein the selected location is a bone fracture and the subject has diabetes and/or osteoporosis.

9. The method of claim 1, wherein the selected location is a bone fracture and the bone fracture is in a limb of said subject that is immobile or paralyzed.

10. The method of claim 1, wherein the selected location comprises a bone implant site.

11. The method of claim 1, wherein the step of providing is performed by injection.

12. The method of claim 1, wherein the aqueous formulation forms a hydrogel having the formula 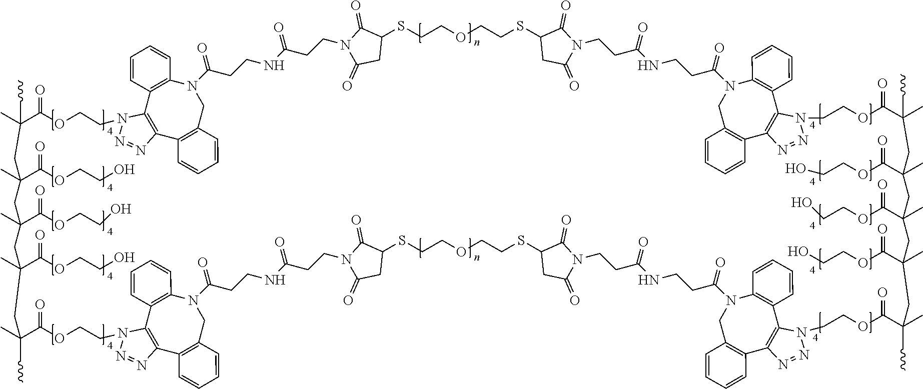
wherein n ranges from 10 to 100 and a wavy line represents a continuation of the polymer backbone.

13. An aqueous formulation, comprising:
linkers having the formula 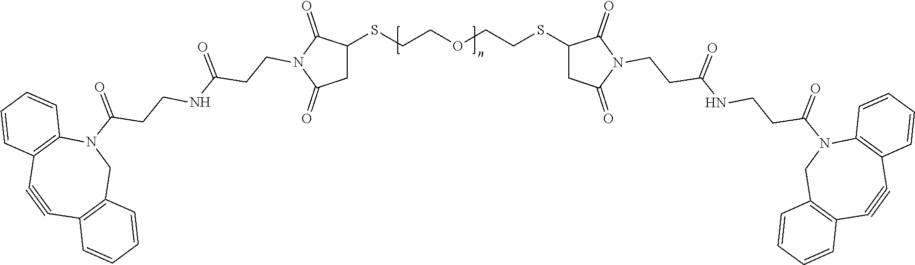
wherein n ranges from 10 to 100, inclusive; and
water-soluble azide functionalized acylate polymers have the general formula 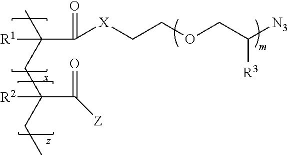
where
R¹ and R² are each independently hydrogen or a C₁ to C6 straight chain, branched chain or cyclic hydrocarbon;
R³ is hydrogen or methyl;
X is —O— or NR⁵— where R⁵ is hydrogen or C₁ to C6 straight chain, branched chain or cyclic hydrocarbon hydrocarbon;
Z is —OR⁶ or NR⁵R⁶ where
R⁵ is hydrogen or C₁ to C6 straight chain, branched chain or cyclic hydrocarbon and
R⁶ is hydrogen, a C₁ to C6 straight chain, branched chain or cyclic hydrocarbon, or a polyethylene glycol chain of two to ten ethylene glycol units;
m is an integer greater than or equal to 1;
x is an integer greater than or equal to 1; and
z is zero or an integer greater than or equal to 1.

14. The aqueous formulation of claim 13, further comprising at least one therapeutic agent.

15. The aqueous formulation of claim 13, wherein n ranges from 65 to 85.

16. The aqueous formulation of any of claim 13, wherein the aqueous formulation forms a hydrogel having the formula 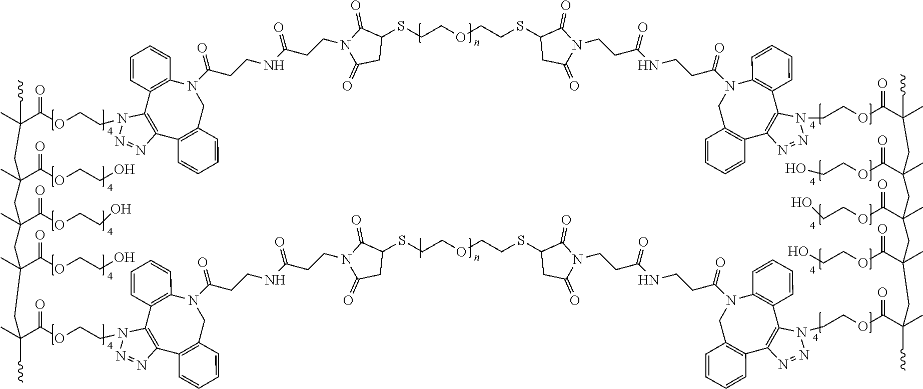
wherein n ranges from 10 to 100 and a wavy line represents a continuation of the polymer backbone.

17. A method of sealing a hole or tear in dura of a mammal, comprising
delivering to the hole or tear in the dura an amount of the aqueous formulation of claim 13, wherein the amount is sufficient to seal the hole or tear.

18. The method of claim 17, wherein the dura is cranial dura or spinal dura.
